(19) United States
(12) Patent Application Publication
(10) Pub. No.: US 2025/0050301 A1
(43) Pub. Date: Feb. 13, 2025

(54) SPATIAL BARCODES BY HYDROGEL LITHOGRAPHY

(71) Applicant: Cellanome, Inc., Foster City, CA (US)

(72) Inventors: Stephen C. MACEVICZ, Bozeman, MT (US); Yir-Shyuan WU, Redwood City, CA (US); Filiz Gorpe YASAR, Palo Alto, CA (US); Tarun Kumar KHURANA, Fremont, CA (US)

(21) Appl. No.: 18/908,638

(22) Filed: Oct. 7, 2024

Related U.S. Application Data
(63) Continuation of application No. PCT/US2023/017896, filed on Apr. 7, 2023.
(60) Provisional application No. 63/329,049, filed on Apr. 8, 2022.

(51) Int. Cl. B01J 19/00; C12N 15/10

(52) U.S. Cl. CPC B01J 19/0046; C12N 15/1065; B01J 2219/00644; B01J 2219/00711; B01J 2219/00722; B01J 2219/00736

(57) ABSTRACT

The invention is directed to methods and systems for producing a surface coated with a plurality of spatial barcodes. In one aspect, beads carrying barcode oligonucleotides are disposed on a surface after which hydrogel chambers are synthesized around each bead and barcode oligonucleotides are released to be captured on the surface in the interiors of the chambers. Captured oligonucleotide barcodes are amplified and optionally sequenced to form a coating of spatial barcodes.

### CROSS-REFERENCE

This application is a continuation of International Patent Application No. PCT/US2023/017896, filed Apr. 7, 2023, which claims the benefit of U.S. Provisional Application No. 63/329,049, filed Apr. 8, 2022, which is herein entirely incorporated by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Molecular tagging has a long history in analytical biochemistry and molecular biology, e.g. Church U.S. Pat. No. 4,942,124; Spitzer et al, Cell, 165(4): 780-791 (2016); Giese, Trends in Analytical Chemistry, 2(7): 166-168 (1983); Hardenbol et al, Nature Biotechnology, 21: 673-678 (2003); Brenner et al, U.S. Pat. No. 7,537,897; Fan et al, Science, 347 (6222): 1258367-1 (2015); Macevicz, U.S. patent publication US2005/0250147; Morris et al, European patent publication 0799897A1; Wallace, U.S. Pat. No. 5,981,179; and the like. Recently, such techniques have been expanded to include the use spatially distributed oligonucleotide barcodes for identifying and studying spatial variations in biological processes, such as tissue-wide gene expression, e.g. Stahl et al, Science, 353(6294): 78-82 (2016); Salmen et al, Nature Protocols, 13: 2501-2534 (2018); Frisen et al, U.S. Pat. No. 9,593,365; and the like. However, cost effective synthesis of spatial barcodes with known sequences, control of spatial barcode distributions, and densities for resolving cellular and subcellular processes has been a challenge, which has been addressed with only partial success by a plethora of different approaches, e.g. Horgan et al, International patent publication, WO/2022/013094; Liu et al, Cell, 183: 1665-1681 (2020); Cho et al, bioRxiv (https://doi.org/10.1101/2021.01.25.427004); Chen et al (https://doi.org/10.1101/2021.01.17.427807); Delly et al, Scientific Reports, 11: 10857 (2021); Rodriques et al, Science, 363(6434): 1463-1467 (2019); and the like. The field of spatial barcode construction would be advanced by the availability of a cost effective spatial barcoding method that permitted flexible synthesis and deposition as well as control over barcoded areas.

### SUMMARY

The invention is directed to a method of coating a surface with spatial barcodes which permit a high degree of control over barcode density and geometry. In one aspect, hydrogel barriers are employed to control the geometry and patterning of spatial barcodes in a process sometimes referred to herein as “hydrogel lithography.”

In as aspect, provided herein is a method of making a spatially barcoded surface, the method comprising: (a) disposing particles on a surface of a channel, wherein the surface is coated with primers for solid phase amplification, and wherein each particle comprises cleavably attached oligonucleotides comprising barcode sequences; (b) synthesizing hydrogel chambers on the surface such that each hydrogel chamber encloses a particle; and (c) cleaving the oligonucleotides from the particles so that the oligonucleotides are released into the chamber such that at least a portion of the oligonucleotides are captured by the primers, thereby obtaining captured oligonucleotides.

In some cases, the method further comprises copying the captured oligonucleotides by extending the primers using the captured oligonucleotides as templates, thereby obtaining copies of the captured oligonucleotides. In some cases, the method further comprises amplifying the copies of the captured oligonucleotides to form clusters. In some cases, the method further comprises sequencing the barcode sequences of the copies of the captured oligonucleotides of the clusters.

In some cases, the copies of the captured oligonucleotides each comprise a sequencing primer binding site adjacent to the barcode sequence, and wherein the method further comprises determining the barcode sequence by a sequencing-by-synthesis method. In some cases, the particles are disposed randomly on the surface. In some cases, the particles are disposed in a regular pattern on the surface. In some cases, the particles are disposed in a rectilinear pattern or a hexagonal pattern on the surface.

In as aspect, provided herein is a method of making a spatially barcoded surface, the method comprising: (a) providing a fluidic device comprising: (i) a channel comprising one or more polymer precursors and a surface comprising capture elements and a plurality of particles disposed on the surface, the particles having cleavably attached oligonucleotides comprising barcodes, (ii) a spatial energy modulation element in optical communication with the surface, and (iii) a detector in optical communication with the surface and in operable association with the spatial energy modulating element, (b) using the detector, detecting each of the plurality of particles and identifying a position thereof on the surface; synthesizing one or more chambers in the channel, each chamber enclosing one or more of the plurality of particles, by projecting light into the channel with the spatial energy modulating element such that the projected light causes cross-linking of the one or more polymer precursors to form polymer matrix walls of the one or more chambers, wherein the polymer matrix walls are substantially impermeable to the oligonucleotides, and wherein positions of the synthesized chambers are determined by the positions of the particles enclosed thereby identified by the detector; and (d) cleaving the oligonucleotides from the particles so that the oligonucleotides are released into the one or more chambers and are captured by the capture elements, thereby obtaining captured oligonucleotides.

In some cases, the method further comprises copying the captured oligonucleotides by extending the capture elements using the captured oligonucleotides as templates, thereby obtaining copies of the captured oligonucleotides. In some cases, the method further comprises performing solid phase amplification of the copies of the captured oligonucleotides on the surface. In some cases, the method further comprises degrading the polymer walls of the one or more chambers.

In another aspect, provided herein is a method of making a spatially barcoded surface, the method comprising one or more cycles of: (a) disposing one or more particles on a surface of a channel, wherein the surface is coated with primers for solid phase amplification, and wherein each particle of the one or more particles comprises one or more cleavably attached oligonucleotides each comprising a barcode sequence; (b) synthesizing hydrogel chambers on the surface such that each hydrogel chamber defines a sub-region of the surface and encloses a particle of the one or more particles; (c) cleaving the one or more oligonucleotides from the one or more particles so that the one or more oligonucleotides are released into the hydrogel chambers such that at least a portion of the one or more oligonucleotides are captured by the primers and copied, thereby obtaining one or more copied oligonucleotides; and (d) degrading the hydrogel chambers, thereby leaving the sub-regions each having attached thereto a copied oligonucleotide of the one or more copied oligonucleotides with a different barcode.

In some cases, the sub-regions do not overlap with one another. In some cases, the method further comprises amplifying the copied oligonucleotides, thereby obtaining amplified oligonucleotides. In some cases, the barcode sequences are determined by sequencing the amplified oligonucleotides. In some cases, a sum of areas of the sub-regions comprises at least 70 percent of an area of the surface. In some cases, the sub-regions form a fractal barcode array. In some cases, each of the sub-regions overlaps at least one other sub-region to form an overlap region. In some cases, the surface in the overlap region comprises at least one oligonucleotide comprising two or more barcode sequences. In some cases, relative positions of the sub-regions on the surface are determined by the oligonucleotides in the overlap regions which comprise two or more barcode sequences. In some cases, the one or more cycles is a plurality of cycles.

In another aspect, provided herein is a composition comprising a substrate with a planar surface comprising a fractal barcode array.

### BRIEF DESCRIPTIONS OF THE DRAWINGS

FIGS. 1A-1E illustrates an embodiment of the invention employing a random disposition of beads on a surface.

FIG. 2 illustrates an embodiment for producing spatially barcoded capture probes.

FIGS. 3A and 3B illustrate another embodiment of the invention employing a regular disposition of beads on a surface.

FIGS. 4A-4D diagrammatically illustrate embodiments of non-overlapping spatial barcode regions.

FIGS. 5A-5E illustrate an embodiment employing overlapping spatial barcode regions.

FIG. 6A shows a distribution of primary barcodes and secondary barcodes in each chamber footprint in a collection of overlapping chamber footprints.

FIG. 6B is a flow chart of an algorithm for assigning barcodes to chamber footprints and their overlaps.

FIGS. 7A and 7B diagrammatically illustrate embodiments of systems for carrying out methods of the invention.

FIGS. 8A and 8B illustrate one embodiment of a flow cell comprising a plurality of channels.

FIG. 9 illustrates steps for preparing cDNAs from single cell target templates, such as mRNA, and its sequencing in an external sequencing instrument after elution from a channel.

FIGS. 10A-10C show a pattern of spatial barcodes produced by a method of the invention.

### DETAILED DESCRIPTION

The practice of the present invention may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation of synthetic polynucleotides, monoclonal antibodies, antibody display systems, cell and tissue culture techniques, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer: A Laboratory Manual; Retroviruses; and Molecular Cloning: A Laboratory Manual (all from Cold Spring Harbor Laboratory Press); Renault and Duchateau, Editors, Site-directed Insertion of Transgenes (Springer, Heidelberg, 2013); Lutz and Bomscheuer, Editors, Protein Engineering Handbook (Wiley-VCH, 2009); and the like. Guidance for selecting materials and components to carry out particular functions may be found in available treatises and references on scientific instrumentation including, but not limited to, Moore et al, Building Scientific Apparatus, Third Edition (Perseus Books, Cambridge, MA); Hermanson, Bioconjugate Techniques, 3rd Edition (Academic Press, 2013); and like references.

The invention is directed to methods of producing a spatially barcoded surface; that is, a surface having attached molecular indicators from which surface position may be determined or indicated. In some embodiments, such molecular indicators comprise surface location information encoded in a polymer sequence, such as, an oligonucleotide sequence. In some embodiments, a plurality of sub-regions of a surface may be formed in which the same unique molecular indicator, or barcode, is present. In other words, in some embodiments, among a plurality of sub-regions, each different sub-region has a different molecular indicator. In some embodiments, a spatially barcoded surface may have sub-regions each with a unique barcode but whose sequences are unknown until used in some process or assay, such as, determining the spatial distribution of gene expression in a tissue. In other embodiments, a spatial barcoded surface may have sub-regions each with a unique barcode whose sequence is determined before use, so that a map of barcode sequences and surface positions exists before use. The present invention encompasses both embodiments. The latter embodiment is advantageous for many applications because it permits molecular processes and/or species to be associated with positions and features of images of the surface.

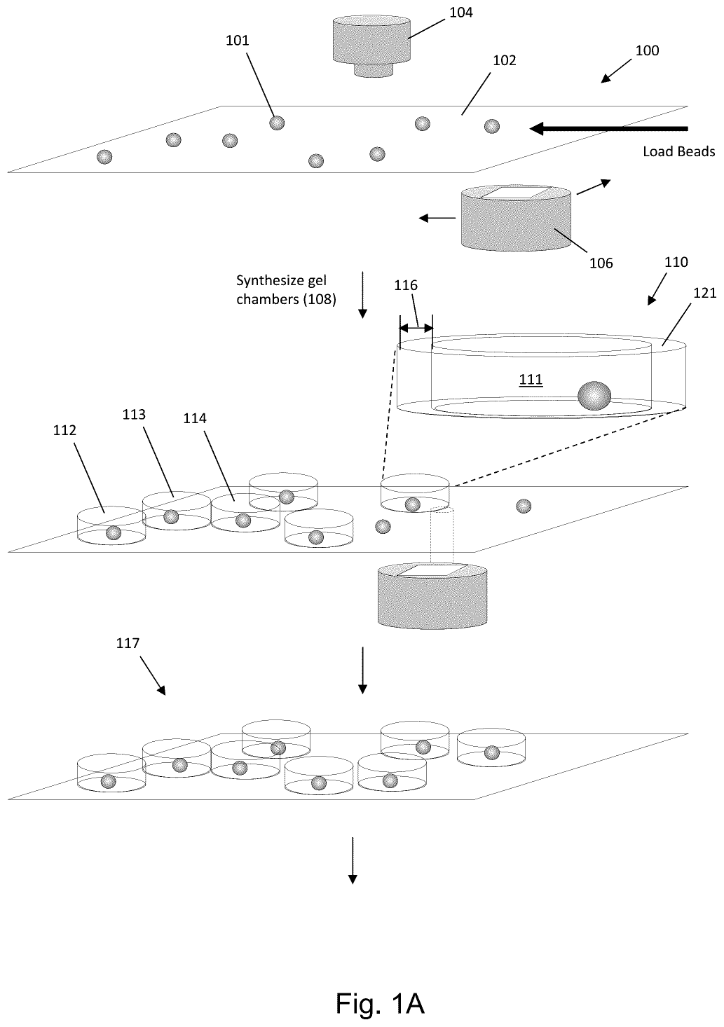

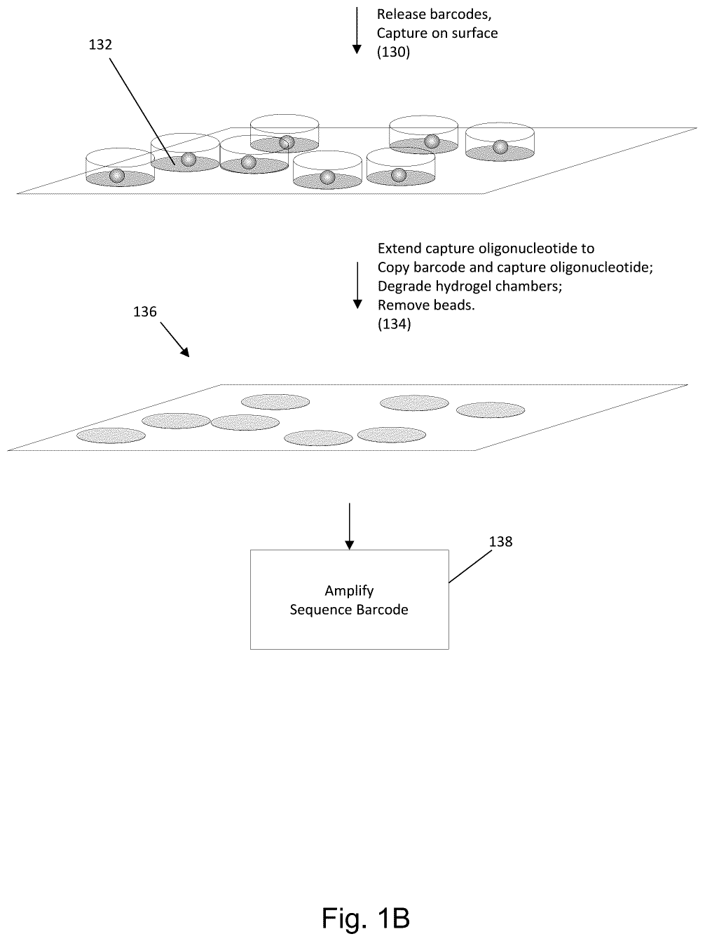

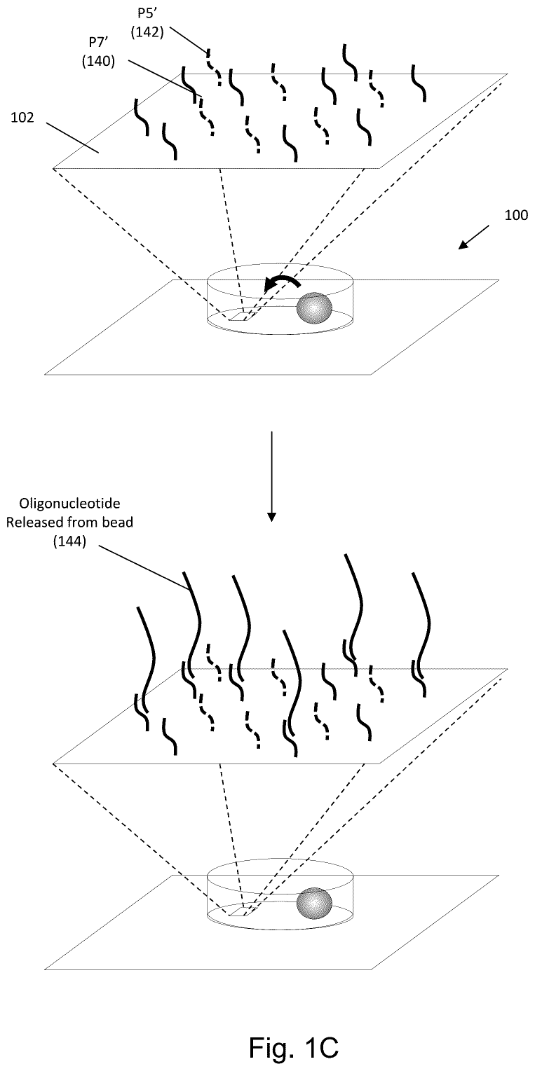

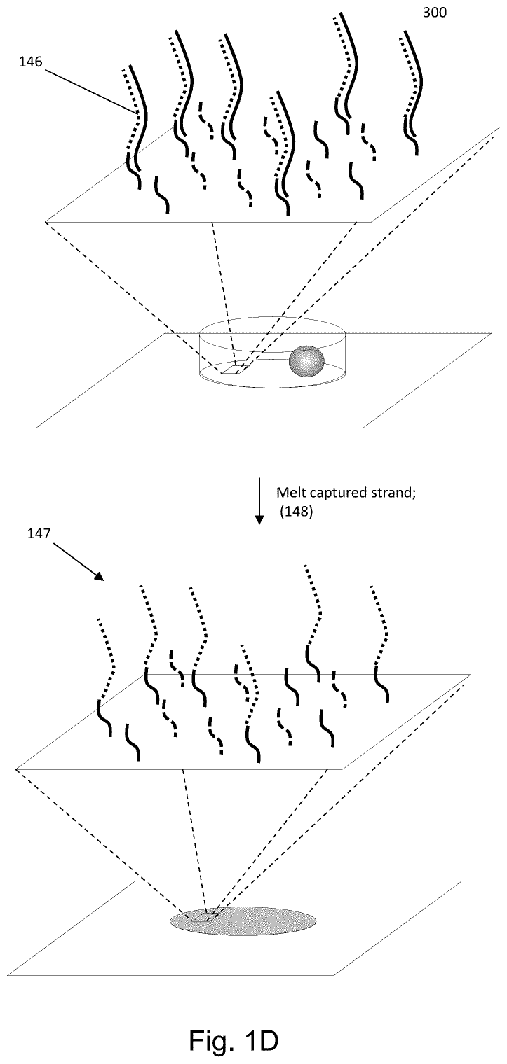

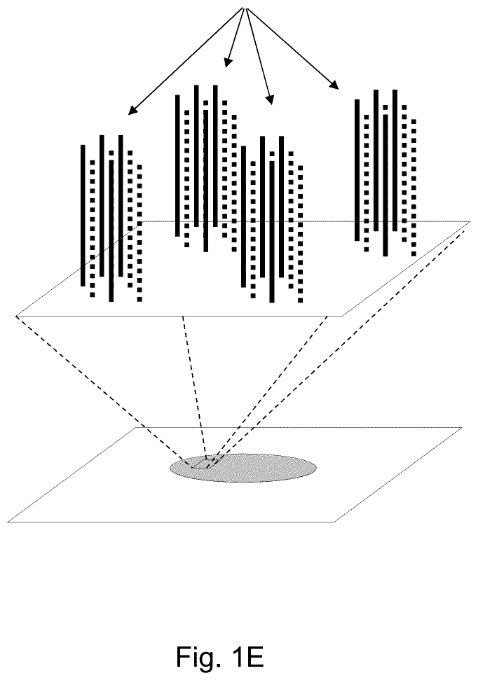

FIGS. 1A and 1B illustrate one embodiment of the method described herein. A fluidics device can be provided having channel (100) with surface (102), detector (104) in optical communication with surface (102) and spatial energy modulating element (106) which may be, for example, a digital micromirror device (DMD), also in optical communication with surface (102) and in operational association with detector (104). A reaction mixture comprising one or more polymer precursors and beads or particles (e.g. 101) can be loaded into channel (100). The beads or particles can have cleavably attached molecular indicators, such as oligonucleotides having at least a segment with a unique nucleotide sequence, or barcode. The terms “bead” and “particle” are used interchangeably to denote a discrete solid support typically used or manipulated in populations but which may be separated from one another. In some embodiments, the term “beads” refers to a monodisperse population of approximately spherical particles typically having diameters with a coefficient of variation less than 25 percent. After loading, the positions of the particles on surface (102) can be determined from optical signals collected by detector (104), which optical signals may comprise an image of all or portions of surface (102). As part of the operational association of detector (104) and spatial energy modulating element (106), particle position information determined from optical signals collected by detector (104) can be translated into instructions transmitted to the spatial energy modulation element (106) to generate and direct light beams into channel (100) to photosynthesize (108) gel barriers, or polymer matrix walls. The gel barriers or polymer matrix walls may be of a predetermined size and shape to form chambers (e.g. 109) around each particle on surface (102).

Blow-up (110) illustrates that the solid appearing structures (112, 113 and 114) can have interiors (111) and walls (121) with a predetermined thickness (116). Likewise, hydrogel chambers can have a predetermined shape (e.g. circular (or annular) with a diameter and an enclosed predetermined area. In the figures, for convenience, chambers are illustrated as standing in isolation without connection with adjacent chambers and as having a cylindrical or annular-like shapes; however, a spatial energy modulating element may synthesize chambers of different shapes and sizes, as is useful for particular applications. In some embodiments of the proliferation assay, each hydrogel chamber synthesized may have the same shape and area, for example, annular-like with an interior area selected from the range of 0.001 to 0.01 mm².

As used herein, “channel” means a container capable of holding fluid (which may be static or flowing) and having at least one surface on which beads may be disposed and chambers synthesized. In some embodiments, a channel may have a first surface and/or a second surface on which chambers may be synthesized and/or on which beads or particles may be disposed. As used herein, reference to a “surface” without reference to “first” or “second” is intended to comprise a first surface or a second surface (if two are present in a fluidics device, such as a flow cell). In some embodiments, a channel may constrain a flow of fluid therethrough from an inlet to an outlet. In other embodiments, a channel may comprise a non-flowing volume of fluid that may be removed, replaced or added to by way of an opening or inlet; that is, in some embodiments, a channel of the invention may be a well or a well-like structure.

Returning to FIG. 1A, after beads on surface (102) are enclosed by chambers (117), the cleavably attached oligonucleotides of the beads can be cleaved and captured by capture elements on surface (102) inside the respective chambers of the beads. The porosity of the polymer matrix walls of the chambers can be selected so that the oligonucleotides released from the beads are effectively restricted to the interior of the chamber into which they are released. Capture elements on surface (102) may comprise uniform coatings (or substantially uniform coatings) of surface-attached oligonucleotides that serve as primers or hybridization binding sites for desired polynucleotides, such as, the oligonucleotides released from the beads. For example, surface (102) may comprise a coating of P5 and P7 primers used for cluster formation in Illumina, Inc. (San Diego) DNA sequencing instruments. In some embodiments, released oligonucleotides are captured (130) on the portions (132) of surface (102) interior to the chambers enclosing beads. After capturing released oligonucleotides, capture oligonucleotides can be extended (134) using the captured oligonucleotides as templates so that the barcodes carried by the released oligonucleotides are copied along with other segments, such as, primer binding sites for sequencing the barcode and segments, e.g. polyT regions, for capturing molecules of interest, such as messenger RNA. The cleavable linkage tethering oligonucleotides to particles or beads may vary widely. Such linkages may include, but not be limited to, cleavable chemical linkages (e.g. base or acid labile), enzymatically cleavable linkage, hybridization (cleavable by heating or treatment with chaotropic reagents (such as formamide)), photochemical cleavage, or the like. In some embodiments, after capture, hydrogel chambers may be degraded, synthesis reagents loaded and captured oligonucleotides copied. In other embodiments, after capture, synthesis reagents are loaded and copying performed prior to chamber degradation and removal of beads. After degrading the hydrogel chambers and removal of beads, a pattern of sub-regions (136) may remain on the surface with each such sub-region seeded with identical copies of barcoded oligonucleotides. The seeded copies may then be amplified to produce larger numbers of surface-bound copies using conventional surface amplification techniques, such as, bridge amplification or the like, e.g. U.S. Pat. Nos. 6,090,592; 6,060,288; 6,787,308; 9,057,097; 9,169,513; 9,476,080; 9,476,080; Adessi et al, Nucleic Acids Research, 28(20): e87 (2000); and the like. In some embodiments, clusters can be formed from the seeded copies by bridge amplification. Such embodiments may further comprise cleaving the ends of the amplified sequences distal to the surface to expose extendable capture sequences.

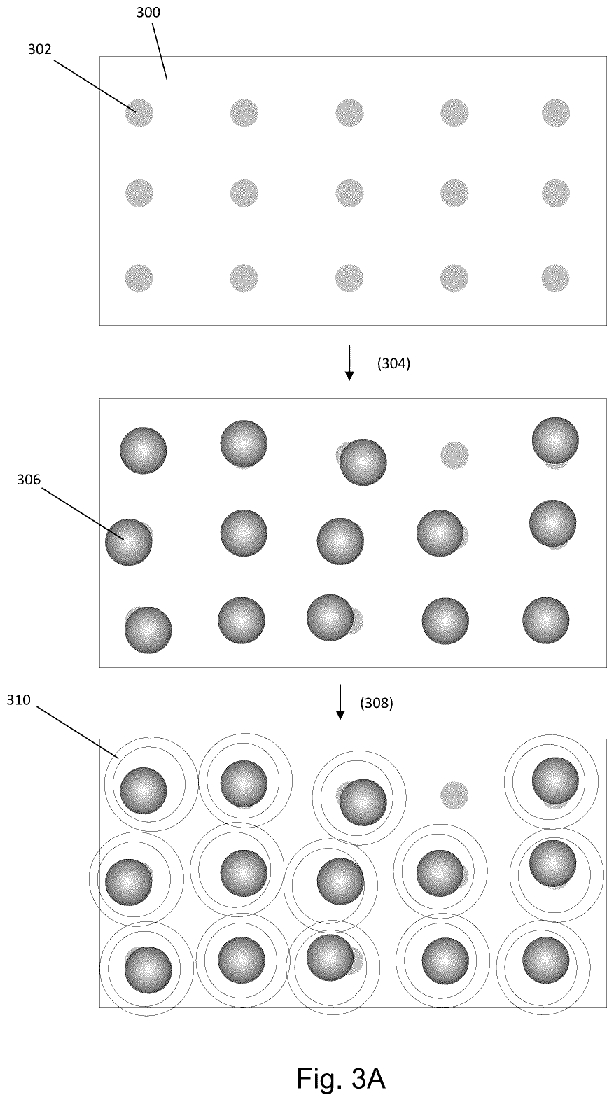

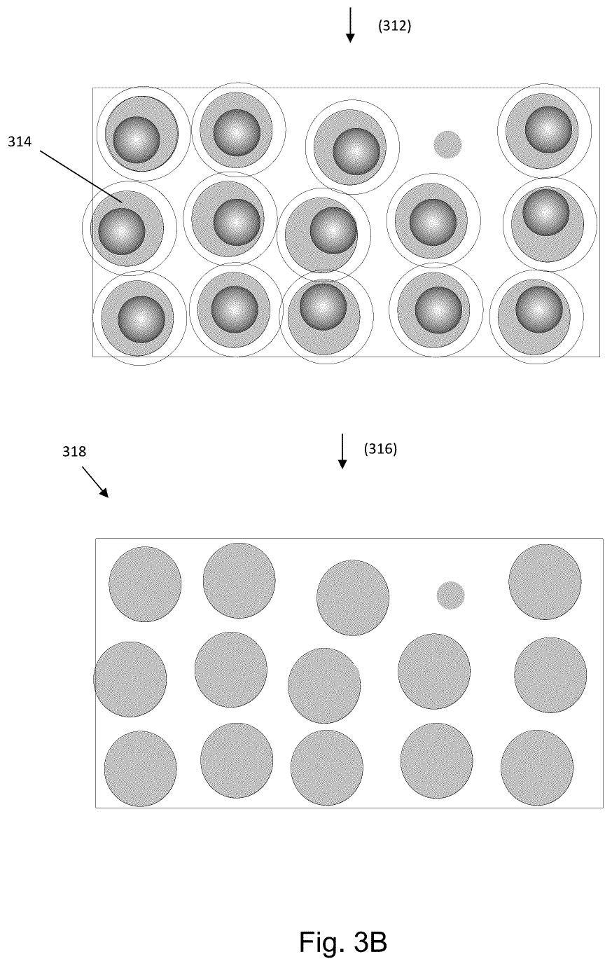

FIGS. 1C-1E comprise a blow-up diagram further describing the embodiment of FIGS. 1A-1B. In the figures, blown-up section (146) of surface (102) of channel (100) shows two types of exemplary capture oligonucleotides, P7′ (140) and P5′ (142) which, in this embodiment, can serve as surface-bound primers for cluster formation by bridge PCR. Capture oligonucleotides can be attached by their 5′ ends so that their 3′ ends may be extended by a polymerase whenever an oligonucleotide released from a bead is captured (e.g. 144). Released oligonucleotides having a P5 sequence on one end may also hybridize and be captured by P5′ complementary primers (142) (not shown in the figure) so that capture oligonucleotide (e.g. 312 of FIG. 3) and other segments are in a reverse orientation with respect to surface (102). In some embodiments, the P5′ primers may be attached to surface (102) by a cleavable linker to remove such sequences (including their extension products) prior to capturing polynucleotides of interest, e.g. mRNAs, e.g. as taught by Cho et al (cited above). After extension of the surface-bound primers to form extension products (146), captured strands can be melted (148) leaving covalently attached seed oligonucleotides (147) from which clusters (150) are be formed. After cluster formation, amplified strands in the undesired orientation may be cleaved or digested as discussed above. Among strands in the desired orientation, sequences distal from surface (102) required for cluster formation may be cleaved to free the capture oligonucleotide for capturing and extension (e.g. see FIG. 3).

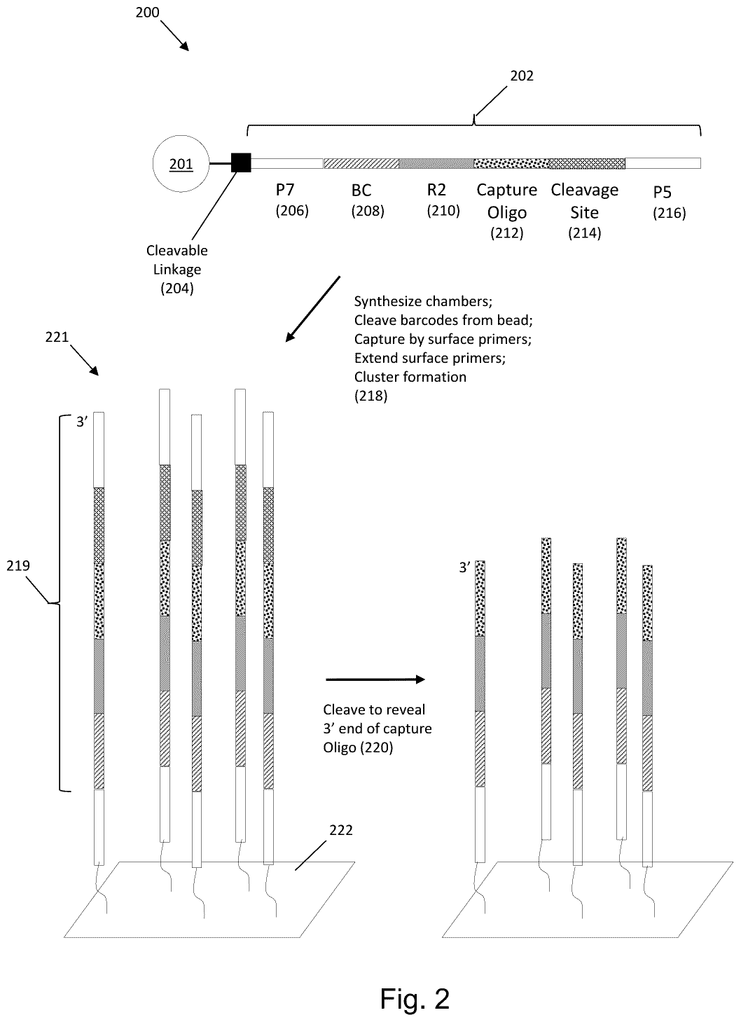

FIG. 2 illustrates an example of a structure of an oligonucleotide attached to a bead or particle for use in the method of the invention. Bead-oligonucleotide conjugate (200) may comprise bead (201) and oligonucleotide (202) cleavably attached to bead (201), for example, by cleavable linkage (204) or by hybridization to an oligonucleotide attached to bead (201). In some embodiments, oligonucleotide (202) may comprise a plurality of segments including, but not limited to, segments at either end for surface amplification (shown as P7 (206) and P5 (216)), a segment with one or more barcodes (208), a primer binding segment (210) for sequencing all or a portion of barcode (208), a capture segment (212), and cleavage site (214) for removing the amplification segment (e.g. P5) so that the end of the capture segment (212) may be extended. Bead or particle (201) may vary widely in regard to size, composition, and loading with oligonucleotide (202). In some embodiments, bead or particle may have a size in the range of 2-500 m in diameter, or in the range of from 4-200 m in diameter, or in the range of from 4-100 m in diameter. In some embodiments, the amount (or loading) of oligonucleotide (202) on bead (201) is sufficient to dispose on the surface bounded by a chamber oligonucleotides (202) in a density in the range of 0.5 to 2 oligonucleotides per 1 μm². As described briefly above, chambers can be synthesized around beads (218), oligonucleotides (202) can be released by cleaving cleavable linkage (204), oligonucleotides (202) can be captured on surface (222) by complementary surface-bound primers, such primers are extended copying oligonucleotide (202), and extension products (219) can be amplified to form clusters (221). After formation of clusters of double stranded oligonucleotides, segments containing the P5 primer binding site may be cleaved (220). For example, whenever capture oligonucleotide (212) is a polyT segment for capturing mRNA, cleavage site (214) may be a DraI endonuclease recognition site.

In some embodiments, beads disposed on a channel surface may be arranged in a regular pattern by modifying the channel surface to have discrete sites to which beads have an affinity. Such affinity may be established by a wide variety of methods including, but not limited to, providing a pattern of hydrophilic sites surrounded by hydrophobic interstitial spaces, hybridization of complementary oligonucleotides, covalent bonding, or the like, e.g. Gopinath et al, Nature, 535: 401-405 (2016), or the like. FIGS. 3A-3B illustrate one embodiment comprising a regular array of beads. In some embodiments, as used herein, “regular array” means a non-random array. In some embodiments, a regular array may be a rectilinear array, a hexagonal array, or the like. FIG. 3A shows surface (300) of a channel having a rectilinear array of sites (302) to which beads (e.g. 306) have an affinity. Upon loading a channel with beads (304), the beads may be attracted to and occupy sites (302). In some embodiments, such occupation may be facilitated by shaking or vibrating the channel. After such loading and placement of the beads on the affinity sites, hydrogel chambers (310) can be synthesized (308) around each bead. The polymer matrix walls of such chamber may be selected (i) to be substantially impermeable to the oligonucleotides once they are cleaved and released from the beads and (ii) to be degradable so that after capture of the released oligonucleotides they may be removed. Captured oligonucleotides may be processed (312) as described above to produce in each chamber a surface coated (314) with unique barcodes. After barcodes are attached to the “floor” of the chamber, the polymer matrix walls may be degraded (316) to produce array (318) with a pattern of spatial barcodes. In some embodiments, such affinity sites or spots have diameters in the range of from 5-500 m or in the range of from 10-1000 m. In some embodiments, such spots or affinity sites are arranged in a rectilinear array, or are arranged in a hexagonal array. In some embodiments, such arrays of such spots or reaction sites have a density in the range of from 10 to 2500 sites/mm², or from 10 to 1000 sites/mm², or from 10 to 500 sites/mm², or from 10 to 100 sites/mm².

In some embodiments, methods described herein may be implemented by (a) disposing particles on a surface of a channel, wherein the surface is coated with primers for solid phase amplification, and wherein each particle comprises cleavably attached oligonucleotides comprising barcode sequences; (b) synthesizing hydrogel chambers on the surface such that each hydrogel chamber encloses a particle; and (c) cleaving the oligonucleotides from the particles so that they are released into the chamber such that a portion of the oligonucleotides is captured by the primers. In some embodiments, methods further comprise copying the captured oligonucleotides by extending primers on the surface using the captured oligonucleotides as templates. In some embodiments, methods further comprise amplifying the copies of the captured oligonucleotides by solid phase, or surface, amplification.

In some embodiments, synthesizing hydrogel chambers is carried out by (a) providing a fluidics device comprising: (i) a channel comprising one or more polymer precursors, a surface comprising capture elements and particles disposed on the surface, the particles having cleavably attached oligonucleotides comprising barcodes, (ii) a spatial energy modulation element in optical communication with the surface, and (iii) a detector in optical communication with the surface and in operable association with the spatial energy modulating element, the detector detecting each of the plurality of particles and determining a position thereof on the surface; and (b) synthesizing one or more chambers in the channel, each chamber enclosing a particle, by projecting light into the channel with a spatial energy modulating element such that the projected light causes cross-linking of the one or more polymer precursors to form polymer matrix walls of the chambers wherein the polymer matrix walls are substantially impermeable to the oligonucleotides and wherein the positions of the synthesized chambers are determined by the positions of particles enclosed thereby identified by the detector.

Exemplary solid phase amplification techniques include, but are not limited to, bridge polymerase chain reaction (bPCR), recombinase-polymerase solid phase amplification (RPA), kinetic exclusion amplification, or the like. Solid phase amplification techniques are disclosed in the following references which are incorporated by reference: Adams, U.S. Pat. No. 5,641,658; Boles, U.S. Pat. No. 6,300,070; Mayer, U.S. Pat. Nos. 7,790,418, 7,985,565, 8,652,810, 9,593,328, 9,902,951 and International patent publication WO1998/44151; Ronaghi, U.S. Pat. Nos. 97,773,268, 9,416,415; 7,763,427, 8,426,134, 7,666,598, 9,309,558; or U.S. Pat. Nos. 6,090,592; 6,060,288; 6,787,308; 9,057,097; 9,169,513; 9,476,080; 9,476,080; Adessi et al, Nucleic Acids Research, 28(20): e87 (2000); and the like.

### Successive Cycles of Bead Loading and Chamber Synthesis

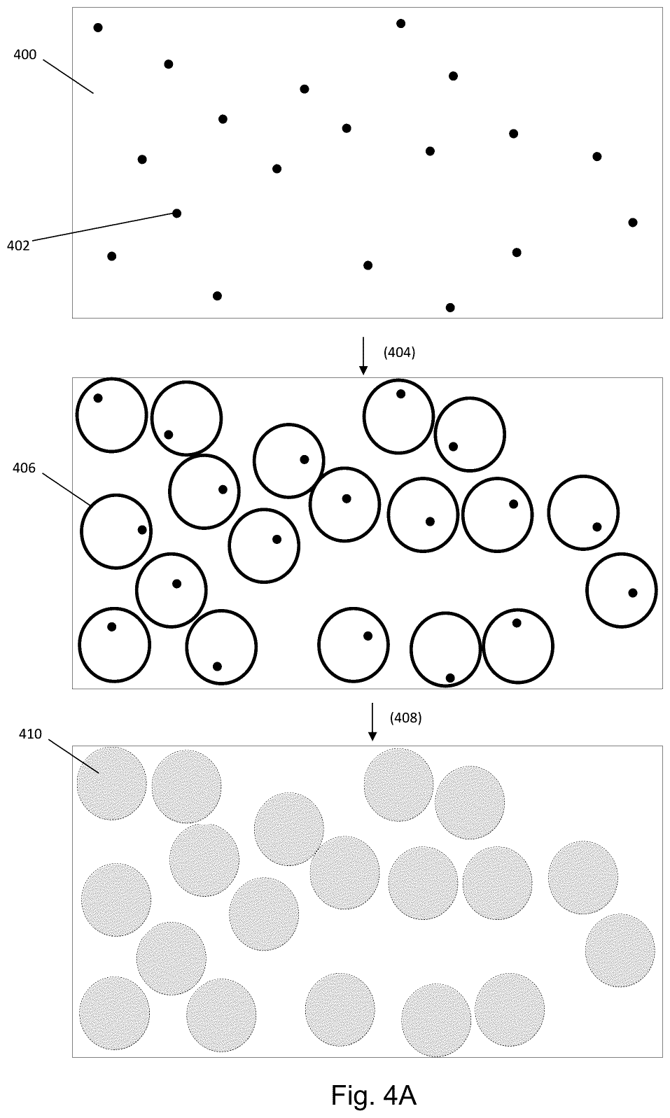

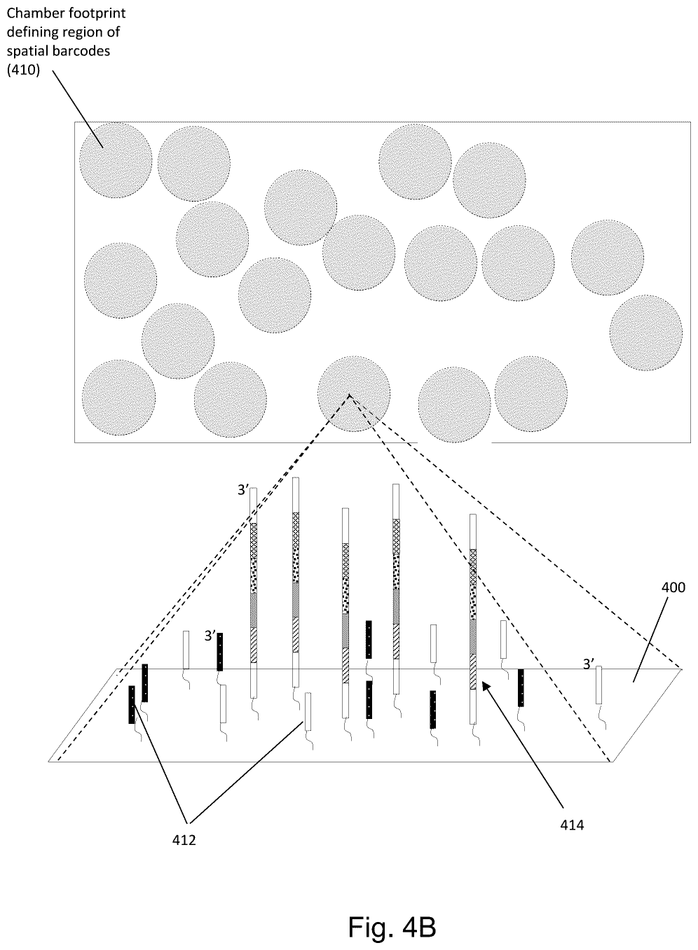

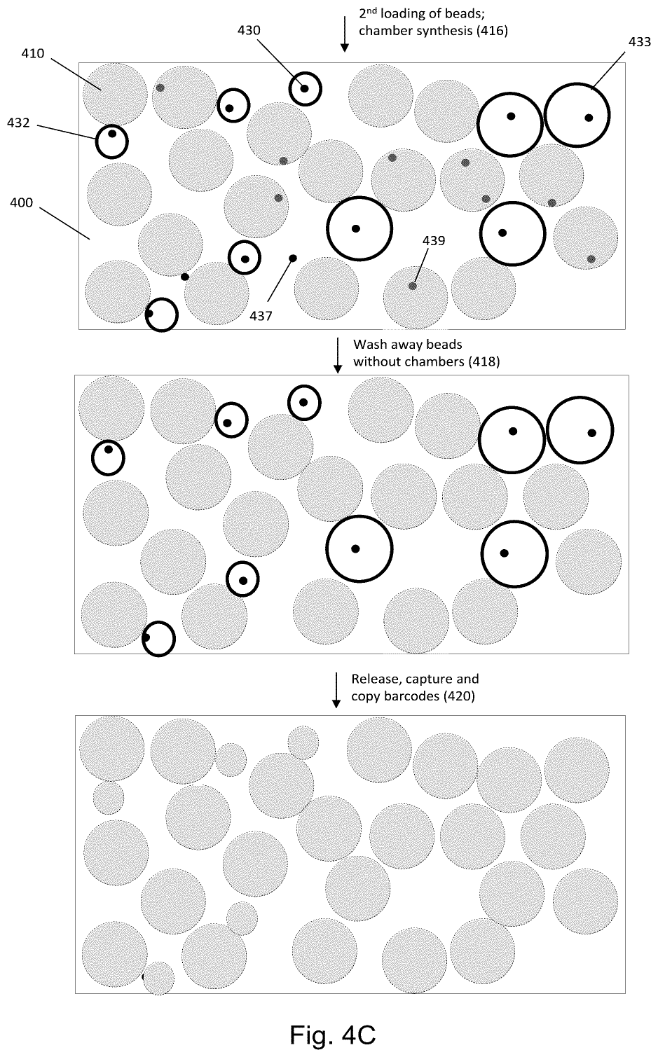

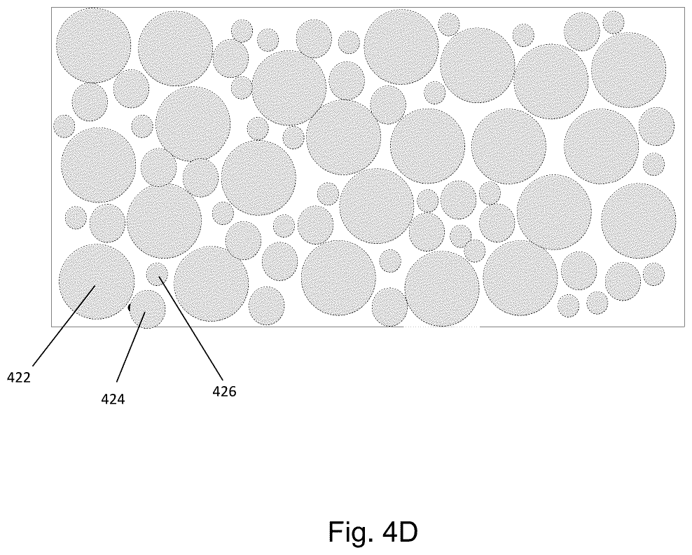

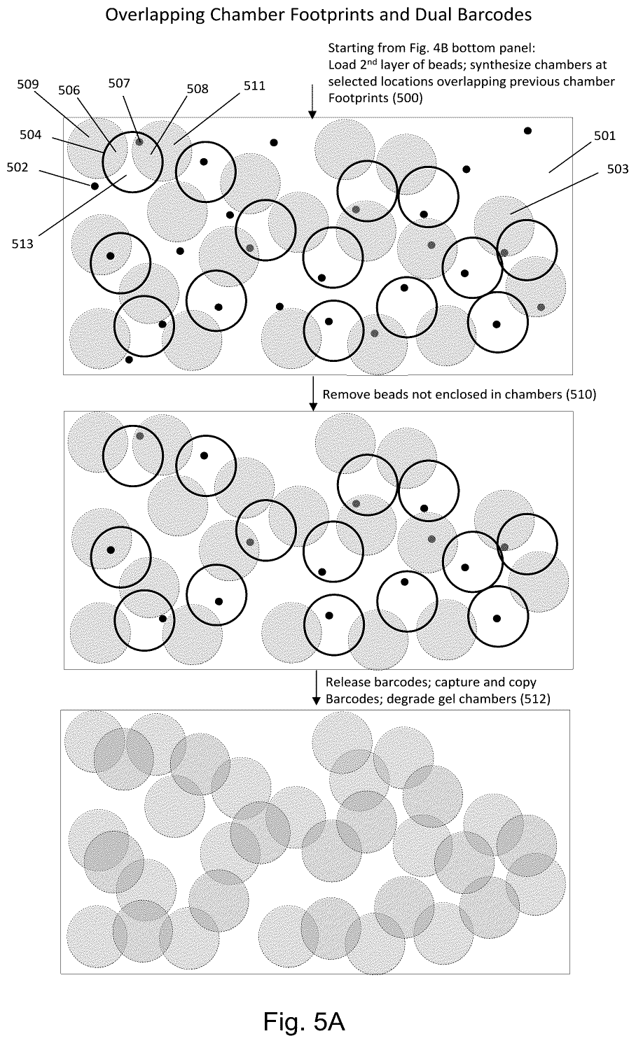

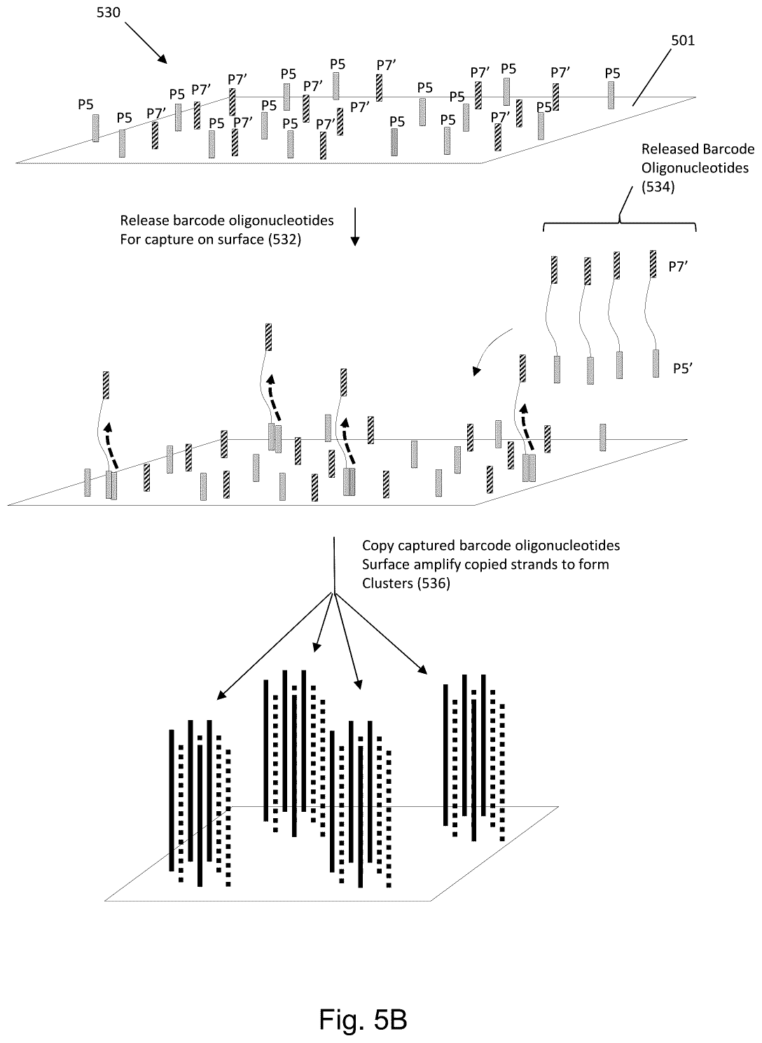

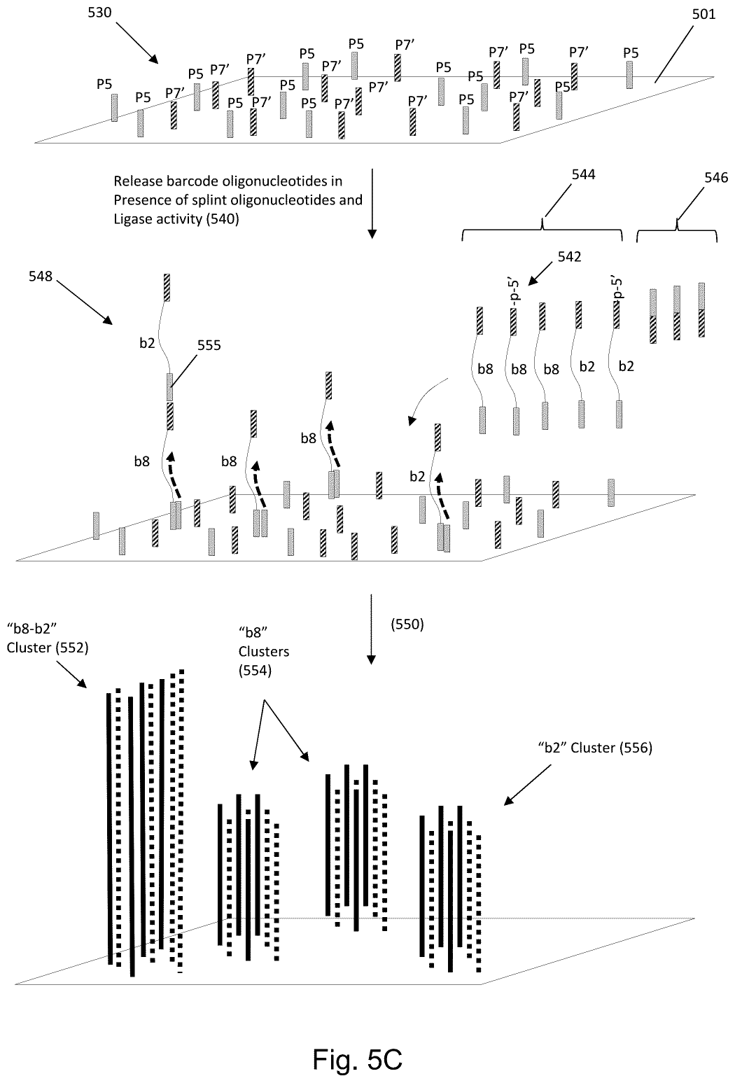

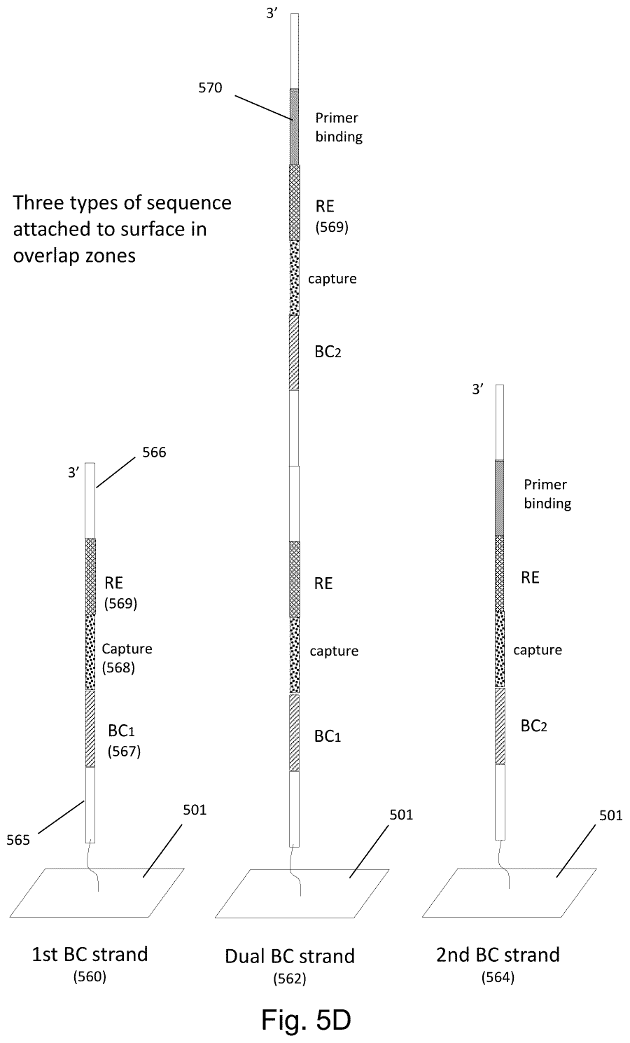

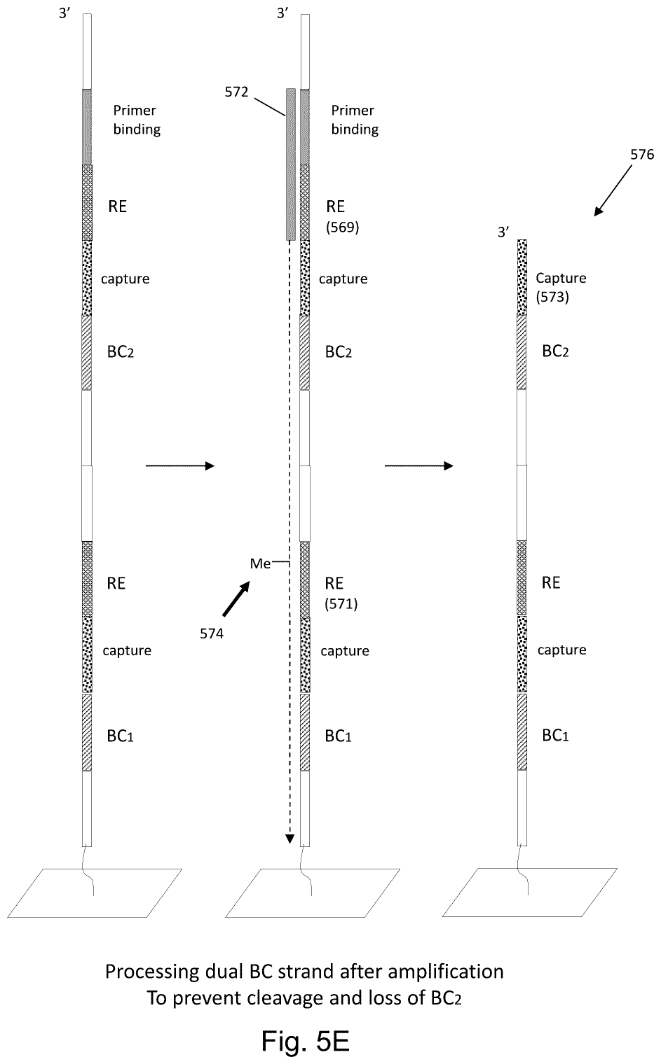

By successive dispositions of beads on a surface followed by chamber syntheses, one may substantially cover an entire surface with spatial barcodes. To this end, one may employ the same or different chamber sizes and/or shapes. In some embodiments wherein beads are disposed randomly on a surface, chambers of the same shape, e.g. annular, but of successively smaller areas may be employed, as illustrated in FIGS. 4A-4C. The upper panel of FIG. 4A shows a random disposition of beads (e.g. 402) on surface (400). Around each bead a chamber (e.g. 406) is synthesized (404). Again, chambers may have a wide variety of shapes. Circular (or annular) shapes are shown in the figures for simplicity). In some embodiments, the position of a chamber around a particular bead may be selected to facilitate more uniform coverage of a surface, to facilitate useful overlaps of chamber footprints in successive cycles, or the like. After chambers are synthesized, barcode-containing oligonucleotides can be released from the beads, after which a portion are captured (by capture oligonucleotides on surface (400)) within the area defined by the chamber (i.e. the chamber footprint). In this example, the capture oligonucleotides are covalently attached to surface (400) by their 5′ ends and their 3′-OH ends are free so that they may be extended with a polymerase using a captured strand as a template. Finally, gel walls of chambers can be degraded and beads are removed (408) leaving surface (400) with regions or zones or chamber footprints (these terms are used interchangeably), e.g. (410), which each have oligonucleotides attached comprising an identical barcode within such zone, region or footprint. Such attached oligonucleotide may further comprise additional elements, such as capture sequences, primer binding sites, and the like, as illustrated in FIGS. 2 and 5C-5D. As shown in FIG. 4B, in some embodiments, within a chamber footprint, surface (400) comprises primers (412) for solid phase amplification, e.g. bridge PCR, and oligonucleotide strands for amplification (414). In some embodiments, especially where beads are randomly disposed on surface (400) when loaded, successive loadings of beads may be made in order to dispose beads in better positions for enclosing in chambers. Likewise, the concentrations of beads in such successive loading may be varied in order to obtain beads in desired positions. In some embodiments, beads may be re-positioned by various means in order to facilitate dispositions of beads more favorable for enclosing in chambers. For example, magnetic beads may be employed, or beads may be moved by optical tweezers, e.g. Hui, Masters Thesis, MIT, Dept. Eng. Comp. Sci. (2001); Grier, Nature, 424: 810-816 (2003); and the like.

As illustrated in FIG. 4B, chamber footprints containing spatial barcodes are randomly distributed and do not cover the entirety of surface (400). Further coverage of a surface may be obtained by implementing successive cycles of bead deposition and chamber synthesis. In some embodiments, chambers of smaller surface area may be employed so that more of the barcode-free interstitial space between current chamber footprints may be filled with additional spatial barcodes. Such embodiment is illustrated in FIGS. 4C and 4D. Onto surface (400) comprising shaded chamber footprints (e.g. 410) a second disposition of beads (e.g. 430) is made, after which further chambers are synthesized around selected bead, which chamber have the same area (e.g. 433) or a lesser area (e.g. 432) than pre-existing chamber footprints (e.g. 410). Beads (e.g. 435) not enclosed by chambers (e.g. 437 or 439) are washed away (418), after which oligonucleotides comprising additional barcodes are released, captured and copied (420). After degrading the gel chambers, the bottom panel of FIG. 4C shows surface (400) with addition area covered with chamber footprints comprising additional spatial barcodes. FIG. 4D shows an embodiment of the above process referred to herein as a “fractal barcode array.” In some embodiments, a fractal barcode array comprises a surface comprising regions, or zones, of successively smaller areas, wherein each zone comprises spatial barcodes comprising the same unique nucleotide sequence and wherein the total area of the zones comprises an area at least 70 percent of the surface, or at least 80 percent of the surface, or at least 90 percent of the surface, or at least 95 percent of the surface. In some embodiments, a fractal barcode array comprises spatial barcode zones of at least three different areas, for example, as illustrated by the areas or zones, (422), (424) and (426) in FIG. 4D. In some embodiments, a fractal barcode array comprises spatial barcode areas having the same shapes. In some embodiments, shapes of spatial barcode areas are circular or polygonal.

### Overlapping Chamber Footprints Having Dual Barcodes

In some embodiments, successive cycles of bead loading and chamber synthesis may result in overlapping spatial barcode areas, or zones, as illustrated in FIG. 5A. In the top panel, surface (501) comprises shaded spatial barcode-containing zones (e.g. 503), for example, as shown in the bottom panel of FIG. 5B. These can be the result of a first cycle of bead disposition, chamber synthesis, release, capture and copying. To such a surface, a second cycle of bead disposition, chamber synthesis, release, capture and copying may be applied, wherein at least a portion of beads are selected for enclosing in chambers because the resulting chambers overlap the areas of one or more previously made chamber footprints, for example, as illustrated by chamber (504) of the upper panel of FIG. 5A. In the figure, the position of second-cycle chamber (504) can be selected to overlap first-cycle chamber footprints (509) and (511) and to enclosed bead (507). Overlap areas (506) and (508) may each comprise dual barcode sequences which, in turn, comprise in a single oligonucleotide strand (i) for (506) a barcode sequence from bead (507) and a barcode sequence already attached to surface (501) in chamber footprint (509) and (ii) for (508) a barcode sequence from bead (507) and a barcode sequence already attached to surface (501) in chamber footprint (511). Structures of the various barcoded strands attached to surface (501) and how they are formed are described above in connection with FIG. 2 and below in connection with FIGS. 5C and 5D. Region (513) enclosed by chamber (504) and between first cycle chamber footprints (509) and (511) can comprise oligonucleotide strands attached to surface (501) each comprising only a barcode sequence from bead (507). After chamber synthesis, non-enclosed beads (e.g. 502) may be removed (510) from surface (501), as shown in the middle panel of FIG. 5A. Barcode containing oligonucleotides can then be released from the enclosed beads, the released sequences are captured by capture oligonucleotides attached to surface (501) and copied (512), or they are ligated to a previously formed first-cycle oligonucleotide strand in an overlap zone to form a dual barcoded oligonucleotide strand, after which the second cycle chambers are degraded. The result is an arrangement of overlapping chamber footprints is shown in the bottom panel of FIG. 5A.

FIGS. 5B and 5C illustrate the formation of single-barcode oligonucleotide strands and dual-barcode oligonucleotide strands on surface (501). FIG. 5B describes with slightly different representations the process previously described in FIGS. 1C-1E. Briefly, surface (501) can have attached a “lawn” (530) of two kinds of primers, designated P5 and P7 (i.e. capture oligonucleotides), attached to surface (501) by their 5′ ends. Barcode oligonucleotides (534) can be released or cleaved (532) from beads and captured by the P5 capture oligonucleotides, after which they can be copied by extending the P5 primers (in the presence of a suitable DNA polymerase and dNTPs). After such copying, the extension products may be surface amplified to give clusters (536).

Dual barcode oligonucleotides may be used to determine the relative positions of chamber footprints on a surface. This is advantageous because the barcodes need be sequenced only once (e.g. after being integrated into a cDNA), rather than twice as is the case with many current spatial barcode methods, e.g. Chen et al, Cell, 185: 1777-1792 (2022); Cho et al, bioRxiv (https://boi.org/10.1101/2021.01.25.427807); Fu et al, Cell, 185: 4621-4633 (2022); Rodriques et al, Science, 363(6434): 1463-1467 (2019); and the like. In some embodiments, these conditions are achieved by delivering to a surface for reaction with barcode oligonucleotides one or more splint oligonucleotides and a ligase activity. A portion of barcode oligonucleotides released from each bead may comprise a 5′ phosphate group that whenever brought into juxtaposition with a 3′ hydroxyl of (for example) a surface-attached barcode oligonucleotide by hybridization to a splint oligonucleotide a phosphodiester bond is formed covalently linking the barcode oligonucleotides. In some cases, self-ligation or ligation to other released barcode oligonucleotides could occur, but the products of such side reactions can be removed by washing and will not affect the method. In some embodiments, the formation of such side products can be adjusted by adjusting the portion of barcode oligonucleotides comprising 5′ phosphate groups, for example. In some embodiments, the portion of barcode oligonucleotides comprising a 5′-phosphate group is in the range of from 0.5 percent to 10 percent; in other embodiments, the portion of barcode oligonucleotides comprising a 5′-phosphate group is in the range of from 1 to 5 percent. These percentages may be conveniently obtained when using the nitrobenzyl photocleavable linkers of Urdea et al (cited above) as the position of the nitrobenzyl moiety in the linker may be selected to produce either a 5′-phosphate or a 5′-hydroxyl. Thus, the two forms of the linker may be used in a proportion corresponding to the desired fraction of barcode oligonucleotides having 5′-phosphate groups. The embodiment of FIG. 5C starts with the same surface (501) and lawn of capture oligonucleotides as the embodiment of FIG. 5B. Barcode oligonucleotides (544), a portion thereof comprising 5′-phosphate groups (542), are released (540) in the presence of splint oligonucleotides (546) on or near surface (501), so that at least some barcode oligonucleotides from adjacent chamber footprints are ligated to form dual barcode oligonucleotides (548). In some embodiments, the splint oligonucleotides may be rendered non-ligatable by the presence of a 3′ blocking group or a dideoxynucleotide at their 3′ ends, i.e. to prevent formation of dimers of splint oligonucleotides and phosphorylated barcode oligonucleotides. In some cases, a dual barcode oligonucleotide may be captured by its internal primer binding site (555), but like the circularized barcode oligonucleotides, such side products will not matter since the desired capture configurations (as shown in FIG. 5C) may be increased or decreased by adjusting reaction conditions. After capture and extension of barcode oligonucleotides (544) and any tandem barcode oligonucleotides formed therefrom, clusters of clonal oligonucleotides comprising barcode sequences can be formed by surface amplification (550). In the overlap regions of adjacent chamber footprints on surface (501) clusters containing three varieties of barcode sequences are formed, exemplary embodiments of which are illustrated in FIGS. 5C and 5D. For example, as illustrated in FIG. 5C, some clusters may comprise dual barcode sequences (552), such as b2 and b8, while other clusters may comprise single barcode sequences (e.g. (554) and (556)) comprising b8 solely and b2 solely, respectively.

FIG. 5D illustrates examples of the three different oligonucleotide strands that may be attached to surface (501) in an overlap zone: first cycle barcode strands (560), second cycle dual barcode strands (562), and second cycle barcode strands (564) (where “cycle” refers to the steps using chambers to define regions where barcodes are attached to a surface). In some embodiments, as described above, oligonucleotide strands containing barcode sequences comprise primer (565) attached by its 5′ end to surface (501) and primer (566) with a free 3′ hydroxyl to permit solid phase amplification, barcode sequence BC1 (567), capture sequence (568) (which for transcriptome analysis may be a polyT segment), and restriction endonuclease site RE (569) (which is used to cleave the distal portion of the oligonucleotide strand so that a free 3′ end of the capture oligonucleotide is produced). The second (or subsequent) cycle oligonucleotide strands may further include primer binding site (570) whenever restriction digestion is employed to produce a capture sequence that may be extended. The restriction sites of the first cycle oligonucleotides and the second cycle oligonucleotides may be the same or different. In some embodiments, the first cycle (or any cycles before the last cycle) may comprise a restriction site for a methylation sensitive endonuclease; that is, an endonuclease that is prevented from cleaving whenever its recognition site is methylated in a specific manner. With such an arrangement, cleavage at only the distal restriction site of dual barcode strands (562) may be accomplished, as illustrated in FIG. 5E. A portion of the sequence of primer (572) is complementary to recognition site (569), so that when annealed to dual barcode oligonucleotides after amplification a non-methylated restriction site is formed. Primer (572) can then be extended with a polymerase in the presence of the four deoxynucleoside triphosphates (dNTPs) with one of the dNTPs substituted with an appropriately methylated analog. Restriction sites in the direction of surface (501) from restriction site (569) (such as restriction site (571)) will be methylated. In some embodiments, type IIs endonuclease, AwlI (New England Biolabs) may be employed for this purpose. In this case, primer (572) would be extended with dATP substituted by the analog N6-methyldeoxyadenosine 5′-triphosphate (which may be prepared as described in Mace, J. Biol. Chem. 259(6): 3616-3669 (1984); and/or Jones et al, J. Am. Chem. Soc., 85(2): 193-201 (1963)). After such extension and digestion the extension product and the cleaved distal segments can be washed away to give dual barcode sequence (576) with distal capture sequence (573) exposed.

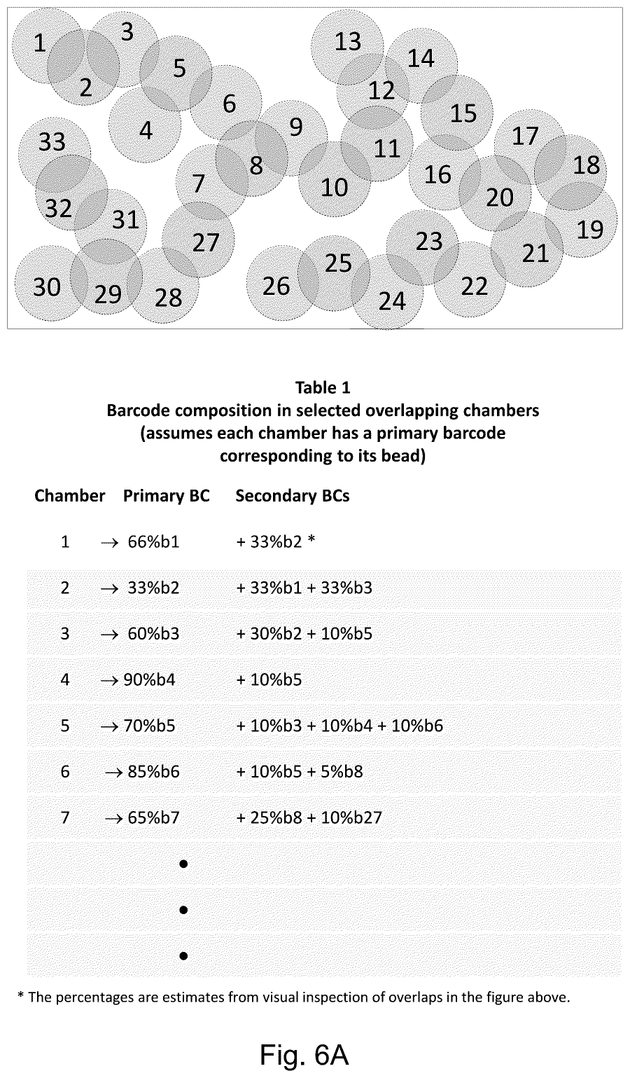

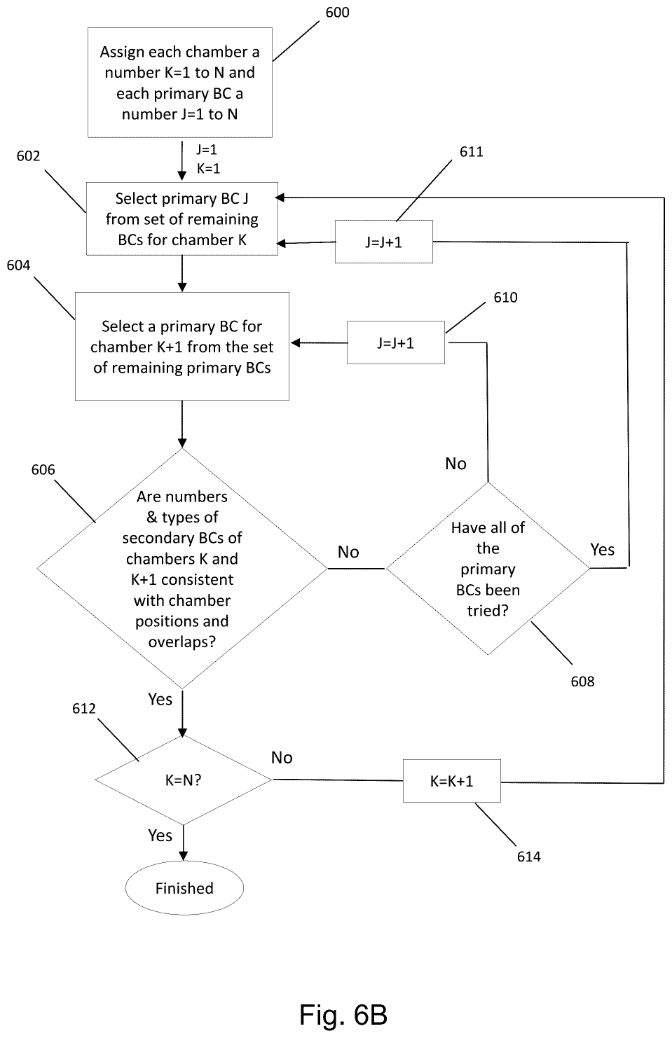

The relative locations of chamber footprints and overlap regions on a surface are known and may be represented as shown in the upper panel of FIG. 6A. (The chamber footprints are arbitrarily labeled 1 through 33). Each chamber can be associated a primary barcode which corresponds to the barcode sequence released from the bead enclosed by the chamber. The problem is to determine which primary barcode is associated with which chamber. Whenever an overlap exists, each chamber with an overlap is also associated with one or more secondary barcodes; that is, barcodes that are part of dual barcode oligonucleotides and derived from a primary barcode of an adjacent and overlapping chamber. A solution to the above problem is the determination of which of the 33 primary barcodes are associated with which chambers in a manner consistent with the types and quantities of single barcode sequences and dual barcode sequences. Table 1 in FIG. 6A provides an example of such data from an inspection of chambers 1 through 7 of the surface represented in the upper panel of the figure. The algorithm represented by the flow chart of FIG. 6B is a systematic use of the tabulated data to find an ordering of primary barcodes in chambers 1-33 that is solution as described above. The chambers are assigned numbers K=1 to N and primary barcodes are assigned numbers J=1 to N. In accordance with steps (602)-(606), starting with chamber K=1, primary barcodes can be selected (from the set of primary barcodes without replacement) for chamber K and K+1 after which types and quantities of secondary barcodes are examined to determine that they are consistent with the current selection of primary barcodes. If they are then consideration is moved to the next chamber (K+2) and the next primary barcode is selected from the remaining primary barcodes. This may continue until no selection of primary barcodes are consistent, in which case the process starts over (608) with the selection of a different primary barcode in chamber 1. The process continues until an ordering of primary barcodes is found that meets the conditions of a solution.

### Systems and Instrumentation

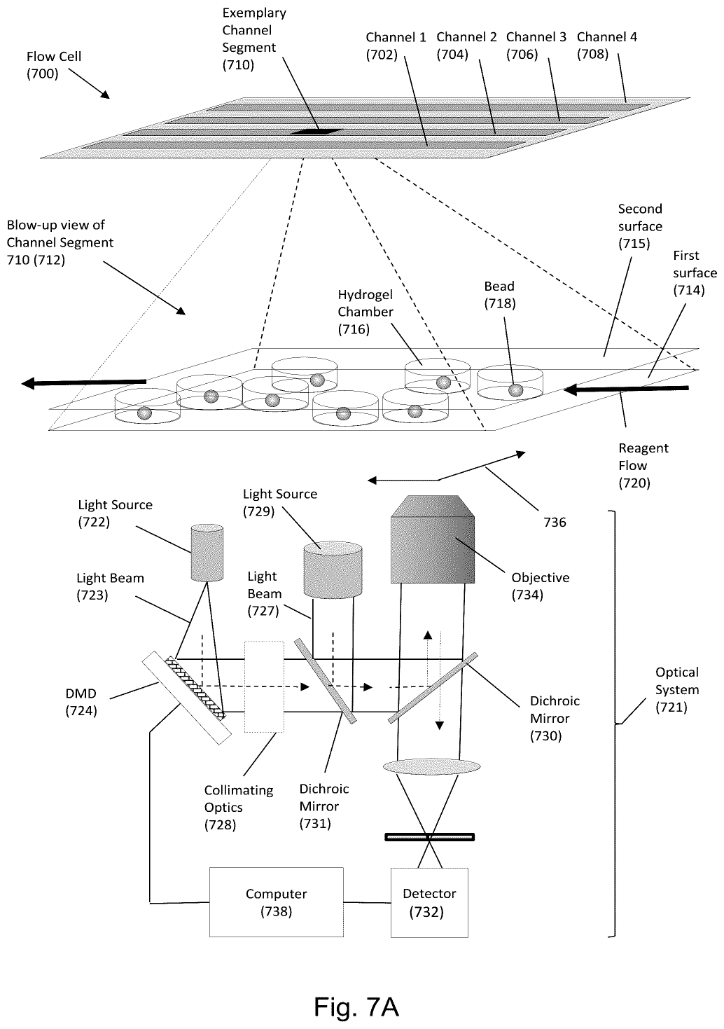

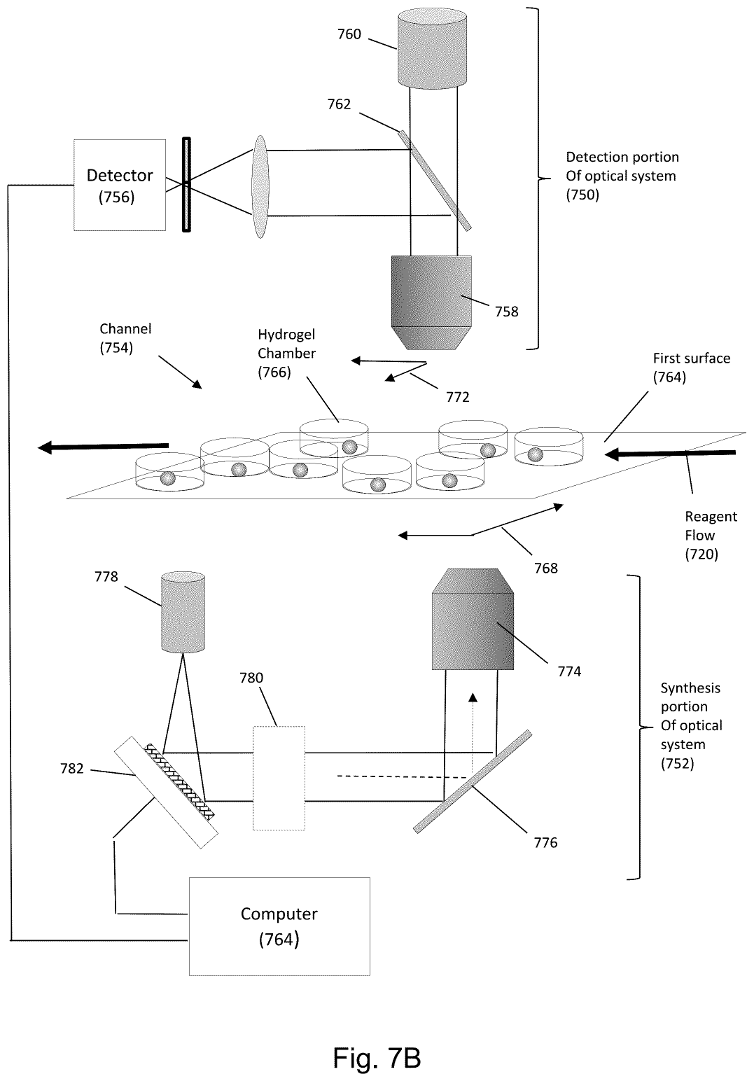

An example of a system for carrying out the above method is illustrated in FIG. 7A. Flow cell (700) can be a component of a fluidic device that provides one or more channels and liquid handling components under programmable control for delivering beads and reagents to the channels. In this illustration, four channels (702, 704, 706, and 708) are shown, with blow-up view (712) of segment (710) of channel 2 (704) shown below. In the abstracted view of flow cell (700) of FIG. 7A, inlets, outlets and other features of the channels are not shown. On first surface (714) of channel 2 (704) a plurality of beads, e.g. (718), can each be enclosed by a hydrogel chamber, e.g. (716). In some embodiments, the porosity of polymer matrix walls of the hydrogel chambers is selected to be impermeable to the beads, but permeable to reagents for forming spatial barcodes. Thus, reagents may be introduced to, and removed from, the interiors of the hydrogel chambers by flowing (720) them through the channels, but beads are retained inside. Below blow-up (712) of channel segment (710) is shown an optical system (721) for photosynthesizing hydrogel chambers at the locations of beads in the channels. One of ordinary skill in the art would recognize that optical systems with different configurations than those of FIGS. 7A and 7B may be employed for carrying out these functions. In some embodiments, one or more DMD-objective subsystems for synthesizing hydrogel structures may be employed to increase the speed of synthesis by synthesizing multiple structures simultaneously.

Returning to FIG. 7A, for photosynthesizing the hydrogel chambers, light source (722) can generate light beam (723) of appropriate wavelength light (e.g. UV light) that passes through an appropriate photo-mask or beam-shaping or beam steering (Galvo) system for shaping a beam to synthesize a desired structure or structures in a channel. In some embodiments, a digital micromirror device (DMD)(724) is employed, in other embodiments, a physical photo-mask may be employed. Chamber position, shape and polymer matrix wall thickness can be determined at least in part from bead position information determined from images collected by detector (732). Reflected light from DMD (724) can be shaped using conventional optics, e.g. collimating optics (728), and is directed through objective lens system (734) into channel 2 segment (710). Objective (734) and flow cell (700) can move relative to one another in the xy-directions (736) to photosynthesize chambers at any position in any of the channels. In some embodiments, flow cell (700) moves and optical system (721) is stationary. In some embodiments, objective (734) may also direct light beam (727) from light source (729) to targets, such as cells, on first surface (714) and collect optical signals, such as fluorescent signals, from assays taking place on first surface (714). Alternatively, optical signal collection may be carried out with a separate objective as shown if FIG. 7B. Information collected by detector (732), or its counterpart in the embodiment of FIG. 7B, particularly cellular positions in their respective channels, can be employed by computer (738) and/or subsidiary controllers to direct DMD (724) and translation devices controlling the relative positions of objective (734) and flow cell (700) to synthesize hydrogel chambers of the appropriate shape and size at the appropriate locations.

FIG. 7B illustrates an alternative optical system in which the detection portion (750) of the optical system moves (772) independently from the movement (768) of the synthesis portion (752) of the optical system. In some cases, detection portion (750) of the optical system comprises detector (756), objective (758), light source (760) and interconnecting optical elements, such as dichroic mirror (762). As with the embodiment of FIG. 7A, detector (756) can be operationally associated with computer (764) and the synthesis portion (752) of the optic system to provide synthesis portion (752) with bead position information. Computer (764) and (738) can also be in operationally associated with stages and/or motors controlling the relative positions of the objectives of the optical systems and the position of the flow cell. In this embodiment, synthesis portion (752) of the optical system is located on the other side of first surface (564) from detection portion (750). As with the embodiment of FIG. 7A, it comprises the conventional components objective (774), mirror (776), collimating optics (780), DMD (782) and light source (778).

In some embodiments, beads, e.g. (718) in FIG. 7A, are disposed randomly on first surface (714). In alternative embodiments, first surface (714) may comprise regularly spaced sites or features for capturing beads so that they are disposed substantially only on such sites or features on the first surface. For example, in some embodiments, such sites or features may be a rectilinear or a hexagonal array of spots.

In some embodiments, systems described herein comprise (a) a channel comprising a first surface, a plurality of beads disposed on the first surface, and one or more polymer precursors; (b) a spatial energy modulating element in optical communication with the first surface; (c) a detector in optical communication with the first surface and in operable association with the spatial energy modulating element, the detector detecting each of the plurality of cells and determining a position thereof on the first surface; and (d) a plurality of gel chambers each gel chamber enclosing a single bead of the plurality of beads, wherein the gel chambers are synthesized by projecting light into the channel with the spatial energy modulating element such that the projected light causes cross-linking of the one or more polymer precursors to form polymer matrix walls of the chambers, wherein the positions of the synthesized chambers are determined by the positions of beads enclosed thereby identified by the detector. It is understood that the term “detector” as used herein may include, but not be limited by, a microscope element that collects and optionally magnifies an image of a portion of a channel and an image analysis element that comprises software for identifying beads and associated position information. A computer element uses such information generated by a detector together with user input to generate commands to other elements, such as, the spatial energy modulating element to carry out a variety of functions including, but not limited to, synthesizing chambers, “on-demand” degrading of chambers, selectively photo-degrading chambers, and the like.

Configurations of such embodiments are illustrated in FIGS. 7A-7B which are described above. In some embodiments, a channel of a fluidic device further comprises a second surface wherein said first surface and the second surface are disposed opposite one another across the channel, and wherein the polymer matrix walls of the chambers extend from the first surface to the second surface to form chambers each having an interior. In some embodiments, chambers in a channel each enclose a single cell. In some embodiments both the first wall and the second wall are made of optically transmissive materials, such as, glass, plastic, or the like, and are positioned so that the first surface and second surface are substantially parallel to one another. The perpendicular distance between a first surface and a second surface may be in the range of from 10 μm to 500 μm, or in the range of from 50 μm to 250 μm.

In some embodiments, cells may be disposed randomly on the first surface. In some embodiments, beads are disposed randomly on the first surface in a Poisson distribution. In some embodiments, in such Poisson distribution beads have a nearest neighbor distance equal to or greater 10 m, equal to or greater 20 m, equal to or greater 30 m, equal to or greater 40 m, equal to or greater 50 m, or equal to or greater 100 am. In some embodiments, a subset of such Poisson distributed beads are each enclosed by an annular-like shaped chamber having a diameter in the range of from 10-500 m. In some embodiments, beads are disposed randomly on the first surface in a Poisson distribution having a density in the range of from 10 to 2500 beads/mm², or from 10 to 1000 beads/mm², or from 10 to 500 beads/mm², or from 10 to 100 beads/mm².

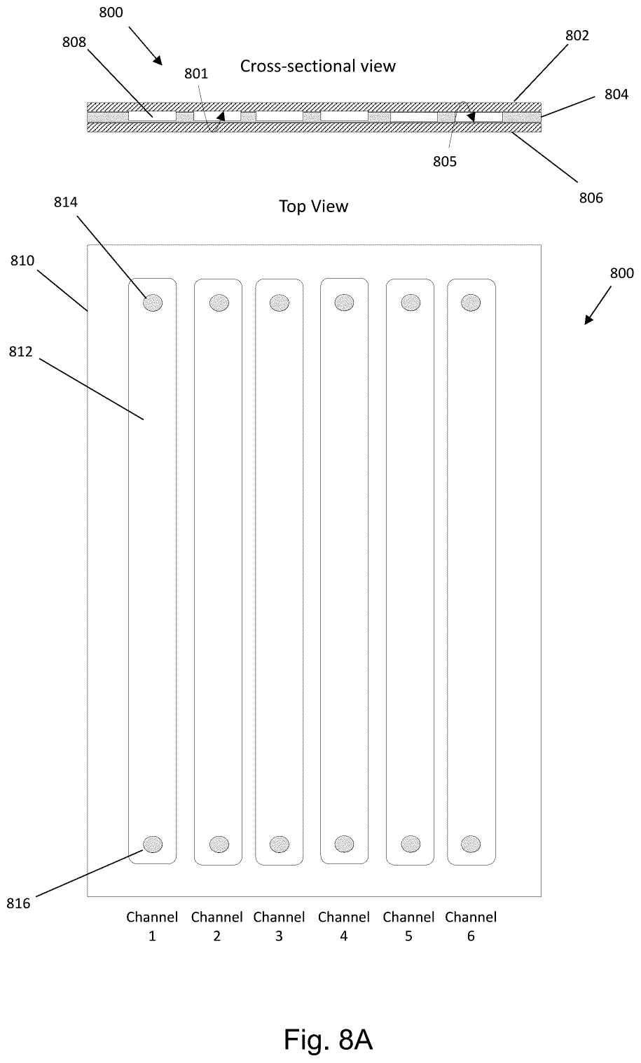

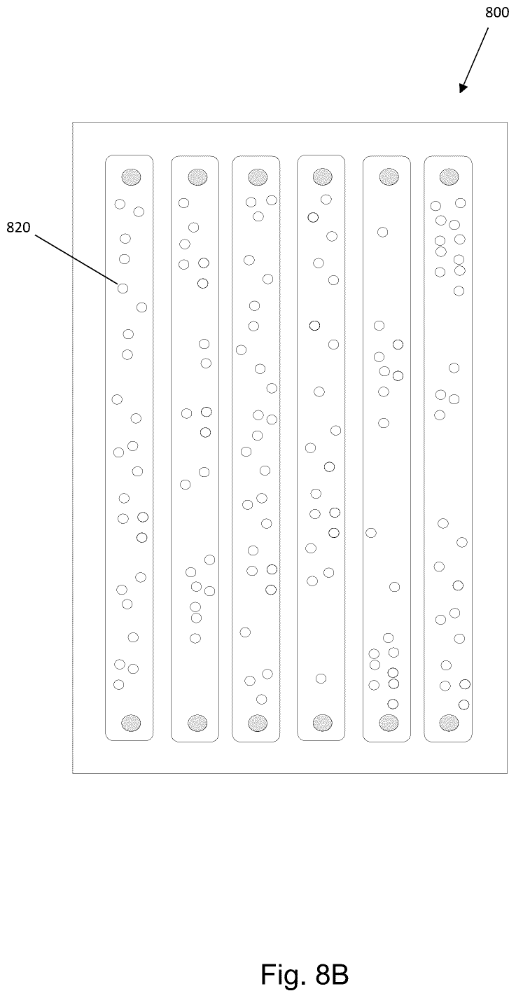

In some embodiments, a plurality of channels may be arranged together in a flow channel as illustrated in FIGS. 8A-8B. In some embodiments, the plurality of channels may be in the range of from 2 to 12, or from 2 to 8, or from 2 to 6, or in the range of from 2 to 4. Flow cell (800) is shown in a cross-sectional view and a top view. Flow cell (800) has bottom, or first, wall (806) with first surface (805); top, or second, wall (802) with second surface (801); and sandwiched sealingly therebetween spacer (804) whose longitudinal holes form channels 1-6, one of which is indicated by (808) in the cross-sectional view, and by (812) in the top view. In some embodiments, spacer (804) may have a thickness in the range of from 10 m to 500 m, or in the range of from 50 m to 250 m, which determines the interior height of the channels. Top wall (802) comprises inlets (814) and outlets (816) for either separately or jointly loading and removing reagents and beads from channels 1-6. In some embodiments, at least one of walls (802) and (806) are made of light transmissive materials, such as glass, plastic, or the like. Flow cell (800) may be operationally associated with a fluidic device that delivers reagents and beads to any of channels 1-6 under programmed control. Guidance for particular designs, including fluid handling and valving for such fluidic systems may be found in U.S. Pat. Nos. 8,921,073; 8,173,080; 8,900,828; and the like, which are incorporated herein by reference. FIG. 8B illustrates channels of flow cell (800) with random distributions (not to scale) of hydrogel chambers with annulus-like cross-sections, such as (820), on their first surfaces.

As noted above, any of first surfaces, second surfaces or polymer matrix wall of chambers may comprise capture elements and other functional groups for carrying out a variety of operations including, but not limited to, capturing beads, capturing analytes (such as, mRNA, secreted proteins, intracellular proteins, or genomic sequences), capturing constituents of analytical reagents (such as, oligonucleotide labels from antibodies), and the like. Derivatizing surfaces for such purposes is well-known to those skilled in the art, as evidenced by the following exemplary references: Integrated DNA Technologies brochure (cited above); Hermanson (cited above); and the like.

As noted above, in some embodiments, a fluidic device of the method comprises or is operationally associated with a detector that either may share an optical path of the spatial energy modulating element or may be disposed adjacent to the second wall or opposite the first wall from the spatial energy modulating element in embodiments, such as wells, that have only a first wall and first surface. The detector is positioned so that it is capable of detecting optical signals from or adjacent to cells in the channel, for example, distributed over the first surface in chambers. In some embodiments, the first and second walls each comprise optically transmissive material, for example, so that a spatial energy modulating element may project light energy to the interior of the channel, and so that a detector may detect optical signals, such as fluorescent emissions or reflected light from biological components. In some embodiments, the projected energy from the spatial energy modulating element is a light energy from a light beam. In some embodiments, the light beam projected by the spatial energy modulating element may have a complex cross-section that permits (in various embodiments) the simultaneous synthesis of a plurality of chambers. Optically transmissive materials include, but are not limited to, glass, quartz, plastic, and like materials.

Spatial energy modulating elements using light energy for polymerization may comprise physical photomasks or virtual photomask, such as, a digital micromirror device (DMD). The following references, which are hereby incorporated by reference, provide guidance in selecting and operating a DMD for photopolymering gels: Chung et al, U.S. patent Ser. No. 10/464,307; Hribar et al, U.S. patent Ser. No. 10/351,819; Das et al, U.S. Pat. No. 9,561,622; Huang et al, Biomicrofluidics, 5: 034109 (2011); and the like.

### Applications

After spatial barcodes are established on a surface in accordance with the invention, oligonucleotide labels, barcodes, genomic fragments, messenger RNAs and similar polynucleotide targets may be sequenced by methods and systems of the invention. In some embodiments, capture elements for this purpose include oligonucleotides attached to a surface in the channel, wherein such oligonucleotides comprise a sequence segment that is complementary to that of the nucleic acids to be captured, which may be polyA segments of mRNAs or an arbitrary “handle” sequence region adjacent to a barcode or oligonucleotide label. A spatial barcode can provide channel position information, and permits externally determined sequences to be associated with individual chambers. In some embodiments, spatial barcodes are present in sufficiently high density such that each chamber covers an area of the first surface that is uniquely associated with one or more spatial barcodes, and usually a single spatial barcode. In some embodiments, the preparation of polynucleotides for a sequencing operation takes place after the target templates (e.g. oligonucleotide label, mRNAs, genomic fragments) are released from cells and captured by complementary sequences in the capture elements. A releasing step depends on the nature of the target templates. For example, oligonucleotide labels attached to antibodies by a disulfide linkage may be released by a reducing agent (which may be the same as a lysing reagent). mRNAs may be release by treating cells with conventional lysing agents. Releasing genomic fragments may require lysing and pre-amplification steps. Lysing conditions may vary widely and may be based on the action of heat, detergent, protease, alkaline, or combinations of such factors. The following references provide guidance for selection of lysing reagents, or lysing buffers, for single-cell lysing conditions for mRNA and/or genomic DNA: Thronhill et al, Prenatal Diagnosis, 21: 490-497 (2001); Kim et al, Fertility and Sterility, 92: 814-818 (2009); Spencer et al, ISME Journal, 10: 427-436 (2016); Tamminen et al, Frontiers Microbiol. Methods, 6: article 195 (2015); and the like. Exemplary lysis conditions include the following: 1) cells in H₂O at 96° C. for 15 min, followed by 15 min at 10° C.; 2) 200 mM KOH, 50 mM dithiotheitol, heat to 65° C. for 10 min; 3) for 4 μL protease-based lysis buffer: 1 μL of 17 μM SDS combined with 3 μL of 125 μg/mL proteinase K, followed by incubation at 37° C. for 60 min, then 95° C. for 15 min (to inactivate the proteinase K); 4) for 10 μL of a detergent-based lysis buffer: 2 μL H₂O, 2 μL 10 mM EDTA, 2 μL 250 mM dithiothreitol, 2 μL 0.5% N-laurylsarcosin salt solution; 5) 200 mM Tris pH7.5, 20 mM EDTA, 2% sarcoyl, 6% Ficoll.

FIG. 9 illustrates a process for capturing a target template and preparing cDNAs for external sequencing. One skilled in the art would recognize that the details of the following examples of target template capture and cDNA synthesis may vary widely depending on the sequencing system employed. In some embodiments, preparation of cDNAs includes a tagmentation step. Guidance for particular embodiments may be found in Picelli et al, Genome Research, 24: 2033-2040 (2014); Bose et al, Genome Biology, 16: 120 (2015); Hashimshony et al, Genome Biology, 17: 77 (2016); Yuan et al, Scientific Reports, 6: 33883 (2016); and like references. Attached to surface (901) by their 5′ ends are oligonucleotides with the following components: primer binding site P7 (for Illumina sequencers) (902), optional primer binding site R1 (for Illumina paired end sequencing), barcode oligonucleotide (906) (which may be or include a spatial barcode), optional unique molecular identifier (908), and capture oligonucleotide (910), which may be a polyT segment whenever mRNA is to be captured. Target template (912) is captured by the hybridization of polyA segment or sequence handle (914) to capture oligonucleotide (910). After capture, capture oligonucleotide (910) and polyA segment (914) are extended by a polymerase (e.g. Moloney murine leukemia virus (MMLV) reverse transcriptase) that leaves a single stranded polyC tail (916). In some embodiments, template switching oligonucleotide (918) is hybridized thereto and the polyC tail is further extended, as show in (930), e.g. Zhu et al, Biotechniques, 30: 892-897 (2001). The unattached strand is melted, the attached strand is amplified, e.g. by a PCR, and eluted for external sequencing (932).

### Hydrogel Chambers

Function. A wide variety of photosynthesizable gels may be used in connection with the invention. In some embodiments, hydrogels are used with the invention in particular because of their compatibility with living cells and the versatility of formulating gels with desired properties including, but not limited to, porosity (which in large part determines what is contained and what is passed by a gel (or polymer matrix) wall, degradability, mechanical strength, ease and speed of synthesis, and the like.

Porosity. In some embodiments, hydrogel porosity is selected to permit passage of selected reagents while at the same time preventing the passage of other reagents or objects, such as, a cell. In some embodiments, hydrogel porosity is selected to prevent the passage of biological cells but to permit the passage of reagents, including proteins, such as polymerases. In some embodiments, such reagents permeable to a polymer matrix wall comprise lysozyme, proteinase K, random hexamers, polymerases, transposases, ligases, deoxynucleotide triphosphates, buffers, cell culture media, or divalent cations. In some embodiments, the at least one polymer matrix comprises pores that are sized to allow diffusion of a reagent through the at least one polymer matrix but are too small to allow DNA or RNA for analysis to traverse the pores (having a size of greater than 100 nucleotides or basepairs, or greater than 300 nucleotides or basepairs). In some embodiments, crosslinking the polymer chains of the hydrogel structure forms a hydrogel matrix having pores (i.e., a porous hydrogel matrix). In some versions, the size of the pores in the hydrogel structures may be regulated or tuned and may be formulated to encapsulate sufficiently large genetic material, such as cells or nucleic acids (e.g., of greater than about 300 base pairs), but to allow smaller materials, such as reagents, or smaller sized nucleic acids (e.g., of less than about 50 base pairs), such as primers, to pass through the pores, thereby passing in and out of the hydrogel structures. In some embodiments, the hydrogels can have any pore size having a diameter sufficient to allow diffusion of the above-listed reagents through the structure while retaining the nucleic acid molecules greater than 500 nucleotides or basepairs in length. In some embodiments, the hydrogel structure can be swollen when the hydrogel is hydrated. The sizes of the pores can then change depending on the water content in the hydrogel of the hydrogel structure. In some embodiments, the pores have a diameter of from about 10 nm to about 100 nm. In some embodiments, the pore size of the hydrogel structures is tuned by varying the ratio of the concentrations of polymer precursors to the concentration of crosslinkers, varying pH, salt concentrations, temperature, light intensity, and the like, by routine experimentation. In some embodiments, the average diameter of pores of a polymer matrix wall prevent passage of molecules having a molecular weight of 25 kiloDaltons (kDa) or greater; or having a molecular weight of 50 kDa or greater; or having a molecular weight of 75 kDa or greater; or having a molecular weight of 100 kDa or greater; or having a molecular weight of 150 kDa or greater.

In some embodiments, DNA or RNA retained have lengths that are sequencable using conventional sequencing-by-synthesis techniques. For example, such DNA or RNA comprise at least 50 nucleotides, or in some embodiments, at least 100 nucleotides. In some embodiments, the pores may have an average diameter from 5 nm to 100 nm. In some embodiments, the pores may have an average diameter from 5 nm to 10 nm, 10 nm to 20 nm, 20 nm to 30 nm, 30 nm to 40 nm, 50 nm to 60 nm, 60 nm to 70 nm, 70 nm to 80 nm, 80 nm to 90 nm, 90 nm to 100 nm. In some embodiments, the pores may have an average diameter larger than 100 nm. In some embodiments, the pores may have an average diameter smaller than 5 nm. The reagent may comprise an enzyme or a primer having a size of less than 50 base pairs (bp). A primer may comprise a single-stranded DNA (ssDNA). In some embodiments, a primer may have a size from 5 bp to 50 bp. In some embodiments, a primer may have a size from 5 bp to 10 bp, 10 bp to 20 bp, from 20 bp to 30 bp, 30 bp to 40 bp, or 40 bp to 50 bp. In some embodiments, a primer may have a size of more than 50 bp. In certain cases, a primer may have a size of less than 5 bp. In some embodiments, the pores may have a diameter from 5 nm to 100 nm. In some embodiments, the pores may have a diameter from 5 nm to 10 nm, 10 nm to 20 nm, 20 nm to 30 nm, 30 nm to 40 nm, 50 nm to 60 nm, 60 nm to 70 nm, 70 nm to 80 nm, 80 nm to 90 nm, 90 nm to 100 nm. In some embodiments, the pores may have a diameter larger than 100 nm. In some embodiments, the pores may have an average diameter smaller than 5 nm. The polymer matrix may have a pore size of about 5 nanometers (nm) to about 100 nm. The polymer matrix may have a pore size of about 5 nm to about 10 nm, about 5 nm to about 20 nm, about 5 nm to about 30 nm, about 5 nm to about 40 nm, about 5 nm to about 50 nm, about 5 nm to about 60 nm, about 5 nm to about 70 nm, about 5 nm to about 80 nm, about 5 nm to about 90 nm, about 5 nm to about 100 nm, about 5 nm to about 110 nm, about 10 nm to about 20 nm, about 10 nm to about 30 nm, about IO nm to about 40 nm, about 10 nm to about 50 nm, about 10 nm to about 60 nm, about 10 nm to about 70 nm, about 10 nm to about 80 nm, about 10 nm to about 90 nm, about 10 nm to about I 00 nm, about 10 nm to about 110 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 20 nm to about 110 nm, about 30 nm to about 40 nm, about 30 nm to about 50 nm, about 30 nm to about 60 nm, about 30 nm to about 70 nm, about 30 nm to about 80 nm, about 30 nm to about 90 nm, about 30 nm to about I 00 nm, about 30 nm to about 110 nm, about 40 nm to about 50 nm, about 40 nm to about 60 nm, about 40 nm to about 70 nm, about 40 nm to about 80 nm, about 40 nm to about 90 nm, about 40 nm to about I 00 nm, about 40 nm to about 110 nm, about 50 nm to about 60 nm, about 50 nm to about 70 nm, about 50 nm to about 80 nm, about 50 nm to about 90 nm, about 50 nm to about 100 nm, about 50 nm to about 110 nm, about 60 nm to about 70 nm, about 60 nm to about 80 nm, about 60 nm to about 90 nm, about 60 nm to about 100 nm, about 60 nm to about 110 nm, about 70 nm to about 80 nm, about 70 nm to about 90 nm, about 70 nm to about 100 nm, about 70 nm to about 110 nm, about 80 nm to about 90 nm, about 80 nm to about 100 nm, about 80 nm to about 110 nm, about 90 nm to about 100 nm, about 90 nm to about 110 nm, or about 100 nm to about 110 nm. The polymer matrix may have a pore size of about 5 nm, about 10 nm, about 20 nm, about 30 nm, about 40 nm, about 50 nm, about 60 nm, about 70 nm, about 80 nm, about 90 nm, about 100 nm, or about 110 nm. The polymer matrix may have a pore size of at least about 5 nm, about 10 nm, about 20 nm, about 30 nm, about 40 nm, about 50 nm, about 60 nm, about 70 nm, about 80 nm, about 90 nm, about 100 nm, or less. The polymer matrix may have a pore size of at most about 10 nm, about 20 nm, about 30 nm, about 40 nm, about 50 nm, about 60 nm, about 70 nm, about 80 nm, about 90 nm, about 100 nm, about 110 nm, or more.

Modulation of Porosity. The pore size in the polymer matrix may be modulated using a chemical reagent, or by applying heat, electrical field, light, or another suitable stimulus. In other words, the polymer matrix may comprise tunable properties (e.g., the pore size) In some cases, the polymer matrix may comprise a thermoresponsive or temperature-responsive polymer. A thermoresponsive polymer (e.g., poly(N-isopropylacrylamide) (NIPAAM)) may phase separate from a solution upon heating or upon cooling (e.g., polymer showing lower critical solution temperature (LCST) or upper critical solution temperature (UCST). The polymer matrix may comprise polymer which may collapse at high temperature in order to, for example, control the pore size of the hydrogel or polymer matrix. Non-limiting examples of thermoresponsive polymers that may be used to form hydrogel/polymer matrix with tunable properties may include Poly(N-vinyl caprolactam), Poly(N-ethyl oxazoline), Poly(methyl vinyl ether), Poly(acrylic acid-coacrylamide), or a combination thereof. A change in temperature may enlarge or contract average pore size in the polymer matrix to allow selected molecules, such as a nucleic acid molecule, a protein, or any biomolecule or molecule smaller than the adjusted pore size to be released from a hydrogel chamber.

Size and Shape of Hydrogel Chambers. In some embodiments, a polymer matrix wall of a chamber inhibits passage of a predetermined component, such as a mammalian cell, genomic DNA, larger polynucleotides (e.g. mRNA greater than 200 ribonucleotides, or greater than 300 ribonucleotides, or 500 ribonucleotides), or the like. In some embodiments, a polymer matrix wall extends from the first surface to a second surface (parallel to the first surface) to form a chamber within a channel. In some embodiments, a chamber has polymer matrix walls and an interior. In some embodiments, the interior of a chamber is sized for enclosing a cell. For example, such chamber may comprise a cylindrical shell or a polygon shell, comprising an inner space, or interior and a polymer matrix wall. In some embodiments, such chambers have annular-like cross-sections. As used herein, the term “annular-like cross-section” means a cross-section topologically equivalent to an annulus. In some embodiments, the inner space, or interior, of a chamber has an inner diameter from 1 μm to 500 μm and a volume in the range of from 1 pico liter to 200 nano liters, or from 100 pico liters to 100 nano liters, or from 100 picoliters to 10 nano liters. In some embodiments, the polymer matrix wall has a thickness of at least 1 μm (micrometer). In some embodiments, the height of a chamber with an annular-like cross section have a value in the range of from 10 m to 500 m, or in the range of from 50 m to 250 m. In some embodiments, a polymer matrix wall having an annular-like cross-section has an aspect ratio (i.e., height/width) of 1 or less. In some embodiments, aspect ratio and polymer matrix wall thickness are selected to maximize chamber stability against forces, such as reagent flow through the channel, washings, and the like. In some embodiments, the at least one polymer matrix wall is a hydrogel wall. In some embodiments, the at least one polymer matrix is degradable. In some embodiments, the degradation of the at least one polymer matrix is “on demand.” In some embodiments, chambers in a channel are non-contiguous. In some embodiments, chambers in a channel may be contiguous with adjacent chambers. In some embodiments, chambers may share polymer matrix walls with one another. In some embodiments, chambers may be synthesized with slits or other orifaces large enough to permit passage of certain components, e.g. beads, but small enough to prevent passage of other components, e.g. cells.

Hydrogel Compositions. In some embodiments, a channel of a fluidic device of a system of the invention comprises one or more polymer precursors for forming chambers. In some embodiments, the one or more polymer precursors comprise hydrogel precursors. Such precursors may be selected from a wide variety of compounds including, but not limited to, polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetraacrylate, or combinations or mixtures thereof In some embodiments, the hydrogel comprises an enzymatically degradable hydrogel, PEGthiol/PEG-acrylate, acrylamide/N,N′-bis(acryloyl)cystamine (BACy), or PEG/PPO. In some embodiments, the following precursors and crosslinker may be used to form chambers with degradable polymer matrix (hydrogel) walls. Polymer precursors may be formed by using any hydrogel precursor and crosslinkers of Table 2 (columns 1 and 3, respectively). The resulting polymer matrices may be degraded with the indicated degradation agents in Table 2 (column 4).

TABLE 2

| | | | Degradation |
|---|---|---|---|
| Precursors | Hydrogels | Crosslinkers | Agents |
| Acrylamide | Polyacrylamide | Bis-acryloyl cystamine | DTT/TCEP/THP |
| | | (structure 1) | |
| PEG-based | PEG | Bis(2-methacryloly)oxyethyl | DTT/TCEP/THP |
| acryloyl | | disulfide (structure 2) | |
| Dextran-based | Dextran | N,N′-(1,2- | NaIO4 |
| acryloyl | | Dihydroxylethylene)bis- | |
| | | acrylamide(structure 3) | |
| Polysaccharide-base | Polysaccharide | Structure 4 | NaOH, |
| acryloyl | | | ethanolamine |
| | | | DTT/TCEP/THP |
| Gelatin-base | Gelatin | Structure 5 | NaOH, |
| acryloyl | | | ethanolamine, |
| | | | nucleophilic bases |
| | | Structure 6 | NaOH, alkali, |
| | | | organic bases |
| | | Structure 7 | Acid |

TABLE 3

| Structure | |
|---|---|
| Number | Formula |
| 1 | 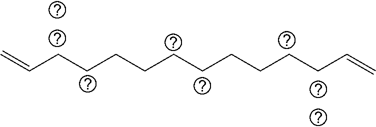 |
| 2 | 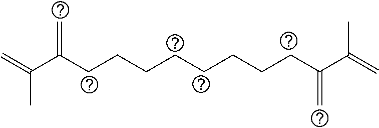 [SMILES: C=C(C)C(=C)CCCCCCCCC(=C)C(=C)C] |
| 3 | 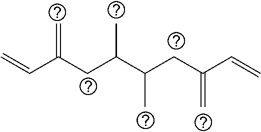 |
| 4 | 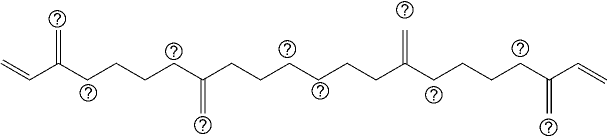 [SMILES: C=CC(=C)CCCCC(=C)CCCCCCC(=C)CCCCC(=C)C=C] |
| 5 | 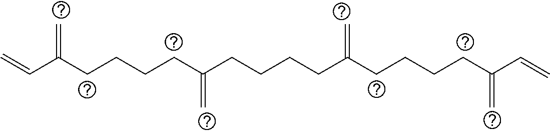 [SMILES: C=CC(=C)CCCCC(=C)CCCCC(=C)CCCCC(=C)C=C] |
| 6 | 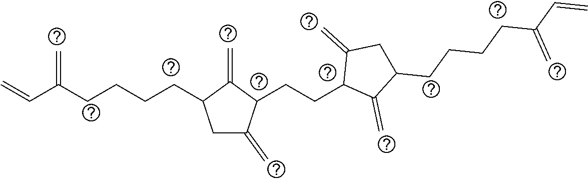 [SMILES: C=CC(=C)CCCCC1CC(=C)C(CCC2C(=C)CC(CCCCC(=C)C=C)C2=C)C1=C] |
| 7 | 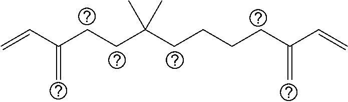 [SMILES: C=CC(=C)CCCCC(C)(C)CCC(=C)C=C] |

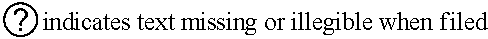

Hydrogel Degradation. In some embodiments, hydrogel chambers of the invention are degradable or depolymerizable either generally within a channel or “on demand” within a channel. Hydrogel chambers that are generally degradable are degraded by treatment with a degradation agent, or equivalently, a depolymerization agent that is exposed to all chambers within channel. Exemplary depolymerization agents include, but are not limited to, heat, light, and/or chemical depolymerization reagents (also sometimes referred to a cleaving reagents or degradation reagents). In some embodiments, on demand degradation may be implemented using polymer precursors that permit photo-crosslinking and photo-degradation, for example, using different wavelengths for crosslinking and for degradation. For example, Eosin Y may be used for radical polymerization at defined regions using 500 nm wavelength, after which illumination at 380 nm can be used to cleave the cross linker. In other embodiments, photo-caged hydrogel cleaving reagents may be included in the formation of polymer matrix walls. For example, acid labile crosslinkers (such as esters, or the like) can be used to create the hydrogel and then UV light can be used to generate local acidic conditions which, in turn, degrades the hydrogel. In some embodiments, the at least one polymer matrix is degradable by at least one of: (i) contacting the at least one polymer matrix with a cleaving reagent; (ii) heating the at least one polymer matrix to at least 90° C.; or (iii) exposing the at least one polymer matrix to a wavelength of light that cleaves a photo-cleavable cross linker that cross links the polymer of the at least one polymer matrix. In some embodiments, the at least one polymer matrix comprises a hydrogel. In some embodiments, the cleaving reagent degrades the hydrogel. In some embodiments, the cleaving reagent comprises a reducing agent, an oxidative agent, an enzyme, a pH based cleaving reagent, or a combination thereof. In some embodiments, the cleaving reagent comprises dithiothreitol (DTT), tris(2-carboxyethyl)phosphine (TCEP), tris(3-hydroxypropyl)phosphine (THP), or a combination thereof. In some embodiments, the surface of the polymer matrix or hydro gel may be functionalized by coupling a functional group to the polymer matrix or hydrogel. Some nonlimiting examples of functional group may include a capture reagent (e.g., pyridinecarboxaldehyde (PCA)), an acrylamide, an agarose, a biotin, a streptavidin, a strep-tag II, a linker, a functional group comprising an aldehyde, a phosphate, a silicate, an ester, an acid, an amide, an aldehyde dithiolane, PEG, a thiol, an alkene, an alkyne, an azide, or a combination thereof. In some cases, the functionalized polymer matrix may be used to capture biomolecules inside a polymer matrix compartment formed adjacent to (e.g., around or on) the biological component. The biomolecule may be produced by the biological component (e.g., secretome from a cell). The functionalized surface of the polymer matrix inside the compartment may be used to capture reagents or molecules from outside the compartment. The functionalized surface may increase surface area covered by a reagent, a molecular sensor, or any molecule of interest (e.g., an antibody).

Partial Degradation. In some embodiments, existing polymer matrix walls may be partially degraded, e.g. to change porosity. In some embodiments, polymer precursors may include degradable beads that form part of, and are embedded in, the polymer matrix walls when synthesized, after which either on-demand or generally, may be degraded, thereby creating an increase in porosity.

Photosynthesis. In some embodiments, the generation of a polymer matrix within said fluidic device comprises exposing the one or more polymer precursors to an energy source. In some embodiments, the energy source is a light generating device. In some embodiments, the light generating device generates light at 350 nm to 800 nm. In some embodiments, the light generating device generates light at 350 nm to 600 nm. In some embodiments, the light generating device generates light at 350 nm to 450 nm. In some embodiments, the light generating device generates UV light. In some embodiments, the generation of a polymer matrix within said fluidic device is performed using a spatial light modulator (SLM) (i.e. a spatial energy modulation element that is capable of generating desired light intensity pattern spatially). In some embodiments, the SLM is a digital micromirror device (DMD). In some embodiments, the SLM is a laser beam steered using a galvanometer. In some embodiments, the SLM is liquid-crystal based.

### Example

### Spatial Barcodes Constructed from a Random

### Disposition of Beads on a Surface

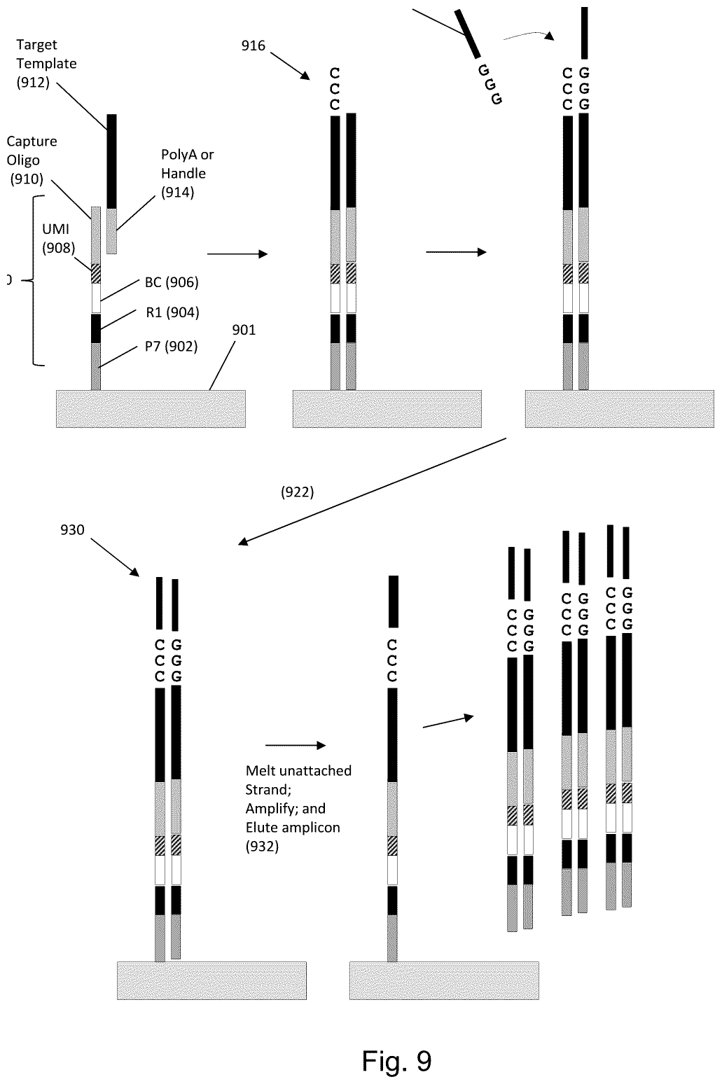

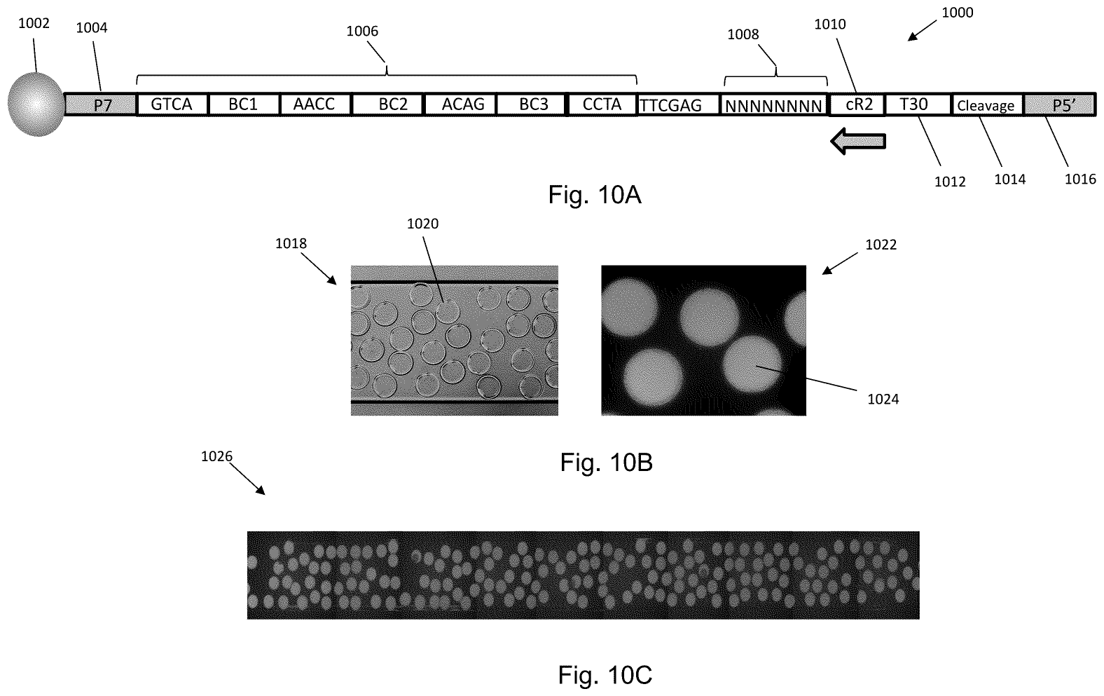

In this example, 10 m beads (streptavidin coated, Bangs Lab Inc., UMC0102) each carrying oligonucleotides with the structure shown in FIG. 10A are loaded into a flow cell channel whose surface comprises a “lawn” of P7 and P5 primers (Illumina sequencing flow cell). After the beads were loaded with polymer precursors, annular (or ring-shaped) hydrogel chambers were synthesized around each bead using apparatus described above. After oligonucleotides were released from the beads, captured and amplified, portions of the barcode sequences were determined by fluorescent-based sequencing-by-synthesis to confirm the distribution and identity of the spatial barcodes within the chambers. Oligonucleotide (1000, FIG. 10A) was attached to bead (1002) by forming a duplex with a shorter biotinylated oligonucleotide (not shown) attached to streptavidin on bead (1002). From 5′ to 3′, oligonucleotide (1000) comprised a P7 primer binding site (1004), a composite barcode segment (1006) containing three barcodes separated by spacer segments, a spacer “TTCGAG”, a random sequence segment (1008), an R2 primer binding site (1010) for sequencing captured and amplified oligonucleotides, a capture oligonucleotide (“T30”, 1012) designed for capturing mRNA, a cleavage site (1014) for removing a distal segment of oligonucleotide (1000) so that a sequence captured by segment (1012) can be extended, and a P5 primer binding site (1016).

The above steps were carried out as follows: A hydrogel precursor solution (40% acrylamide gel with 30 mg/mL photoinitiator (3× lithium phenyl-2,4,6-trimethylbenzoylphosphinate)) containing 10 um beads with barcode oligonucleotides attached by hybridization were loaded into a flow cell channel. Photosynthesis of chambers was performed with a DMD programmed to project a ring-shaped beam with 200 uM in radius around each of the beads. After gel structures were formed, excess precursor solution was washed with 600 ul of 1×PBS. Barcode oligonucleotides were released (or melted) from the beads by loading the flow cell with a releasing solution consisting of 50% formamide in a 350 mM NaCl solution, after which the flow cell was heated to 70° C. The barcode oligonucleotides from each bead were confined to their respective hydrogel chambers and captured by anchored P7 primers. A Bst polymerase mix was then loaded to the flow cell and the P7 primers extended using the captured barcode oligonucleotides as templates, after which clusters were formed from the extended sequences by bridge amplification. Sequencing primers were annealed to the amplified sequences and barcode sequences identified. FIG. 10B shows on the left a magnified segment of a flow cell channel (1018) in which individual beads have been enclosed by hydrogel chambers, e.g. (1020). On the right is a magnified segment (1022) of flow channel (1018) after incorporation of a fluorescently labeled nucleotide in the sequencing of a barcode segment of the amplified oligonucleotides. The distribution of fluorescence in the chambers (e.g. 1024) shows a uniform coating of spatial barcodes within the interiors of the chambers. FIG. 10C shows a larger segment of a flow cell channel (1026) with a distribution of spatial barcodes.

While the present invention has been described with reference to several particular example embodiments, those skilled in the art will recognize that many changes may be made thereto without departing from the spirit and scope of the present invention. The present invention is applicable to a variety of sensor implementations and other subject matter, in addition to those discussed above.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immuology, 6^{th} edition (Saunders, 2007).

“Assay,” in some embodiments, refers to a process for detecting or measuring a cellular characteristic or property of single cells or of a population of cells. Typically process steps of an assay comprise a chemical, biochemical or molecular reaction (such as a cleavage of a bond, specific binding of complementary components, enzyme reactions, dissolution of complementary components, or the like) or a change of physical state (such as an increase or decrease in temperature, change in energy level, or the like) and result in the generation of a signal (or signals) from which the presence, absence or magnitude of a quantity related to a cell may be inferred. The nature of the signal produced by an assay may vary widely and can include, but is not limited to, an electrical signal, an optical signal, a chemical signal, or a material signal. A material signal comprises the production of a material that comprises information that can be extracted. For example, a material signal may be the amplification of a polynucleotide whose length, quantity, composition, or nucleotide sequence is indicative of a cellular characteristic. For example, a barcode oligonucleotide may be a material signal. Characteristics or properties of cells that are detected or measured may vary widely and include, but are not limited to, cytotoxicity, viability, proliferation capacity under selected conditions, size, shape, motility, types and profiles of cell surface, or cell membrane proteins, types and profiles of secreted proteins, production of metabolites, transcriptome, gene copy numbers, gene or allele identity, chromatin accessibility profiles, vector copy numbers for engineered or infected cells, and the like. Assays of special interest for cell-based therapy include, but are not limited to, cytotoxicity, viability, activation, proliferation capacity under selected conditions, chromatin accessibility profiles, types and profiles of cell surface or membrane proteins, types and profiles of secreted proteins, intracellular proteins, transcriptome, vector copy number, and the like.

“Barcode” means a molecular label or identifier. In some embodiments, a barcode is a molecule attached to an analyte or a segment of an analyte (for example, in the case of polynucleotide barcodes and analytes) which may be used to identify the analyte. In some embodiments, a barcode (referred to herein as a “spatial barcode”) may be attached to a surface to identify a location on the surface. In some embodiments, populations of identical spatial barcodes may be disposed within a particular area on a surface. In some embodiments, there may be a one-to-one correspondence between different spatial barcodes and different areas on a surface; that is, each different area has a different and unique barcode. In some embodiments, the identity of a spatial barcode is determinable, for example, by sequencing whenever a spatial barcode is a polynucleotide. In some embodiments, a spatial barcode is an oligonucleotide. In some embodiments, an oligonucleotide spatial barcode comprises a random sequence oligonucleotide. A random sequence oligonucleotide is typically synthesized by a “split and mix” synthesis techniques, for example, as described in the following references that are incorporated herein by reference: Church, U.S. Pat. No. 4,942,124; Godron et al, International patent publication WO2020/120442; Seelig et al, U.S. patent publication 2016/0138086; and the like. Sometimes a random oligonucleotide is represented as “NNN . . . N.” In some embodiments, the term “barcode” includes composite barcodes; that is, an oligonucleotide segment that comprises sub-segments that identify different objects. For example, a first segment of a composite barcode may identify a particular area of a surface and a second segment of a composite barcode may identify a particular molecule (a so-called “unique molecular identifier” or UMI).

“Cells” refers to biological cells that may be assayed by methods and systems of the invention comprise, but are not limited to, vertebrate, non-vertebrate, eukaryotic, mammalian, microbial, protozoan, prokaryotic, bacterial, insect, or fungal cells. In some embodiments, mammalian cells are assayed by methods and systems of the invention. In particular, any mammalian cell which may be, or has been, genetically altered for use in a medical, industrial, environmental, or remedial process, may be analyzed by methods and systems of the invention. In some embodiments, “cells” as used herein comprise genetically modified mammalian cells. In some embodiments, “cells” comprise stem cells. In some embodiments, “cells” refer to cells modified by CRISPR Cas9 techniques. In some embodiments, “cells” refer to cells of the immune system including, but not limited to, cytotoxic T lymphocytes, regulatory T cells, CD4+ T cells, CD8+ T cells, natural killer cells, antigen-presenting cells, or dendritic cells. Of special interest are cytotoxic T lymphocytes engineered for therapeutic applications, such as cancer therapy.

“Cleavable linkage” or “cleavable nucleotide” means any of wide variety of cleavable linkages, or more particularly, cleavable nucleotides, may be used with embodiments of the invention. As used herein, the term “cleavable site” refers to a nucleotide or backbone linkage of a single stranded nucleic acid sequence that can be excised or cleaved under predetermined conditions, thereby separating the single stranded nucleic acid sequence into two parts. In some embodiments, a step of cleaving a cleavable nucleotide or a cleavable linkage leaves a free 3′-hydroxyl on a cleaved strand, thereby, for example permitting the cleaved strand to be extended by a polymerase. Cleaving steps may be carried out chemically, thermally, enzymatically or by light-based cleavage. Sometimes the term “releasing” may be used in reference to cleaving an oligonucleotide label, for example, by a releasing reagent or agent, which may be one or more of those listed above. In some embodiments, cleavable nucleotides may be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavage by glycosylases and/or endonucleases may require a double stranded DNA substrate. Methods synthesizing and cleaving nucleic acids containing chemically cleavable, thermally cleavable, and photo-labile groups are described for example, in U.S. Pat. No. 5,700,642, which is incorporated herein by reference. Further cleavable linkages are disclosed in the following references: Pon, R., Methods Mol. Biol. 20:465-496 (1993); Verma et al., Ann. Rev. Biochem. 67:99-134 (1998); U.S. Pat. Nos. 5,739,386, 5,700,642 and 5,830,655; and U.S. Patent Publication Nos. 2003/0186226 and 2004/0106728, Urdea et al, U.S. Pat. No. 5,367,066, which are incorporated herein by reference. Synthesis and cleavage conditions of chemically cleavable oligonucleotides are described in U.S. Pat. Nos. 5,700,642 and 5,830,655. Phosphorothioate internucleotide linkage may be selectively cleaved under mild oxidative conditions. Selective cleavage of the phosphoramidate bond may be carried out under mild acid conditions, such as 80% acetic acid. Selective cleavage of ribose may be carried out by treatment with dilute ammonium hydroxide. In another embodiment, a cleavable linking moiety may be an amino linker. The resulting oligonucleotides bound to the linker via a phosphoramidite linkage may be cleaved with 80% acetic acid yielding a 3′-phosphorylated oligonucleotide, which may (if desired) be removed by a phosphatase. In some embodiments, the cleavable linking moiety may be a photocleavable linker, such as an ortho-nitrobenzyl photocleavable linker. Synthesis and cleavage conditions of photolabile oligonucleotides on solid supports are described, for example, in Venkatesan et al., J. Org. Chem. 61:525-529 (1996), Kahl et al., J. Org. Chem. 64:507-510 (1999), Kahl et al., J. Org. Chem. 63:4870-4871 (1998), Greenberg et al., J. Org. Chem. 59:746-753 (1994), Holmes et al., J. Org. Chem. 62:2370-2380 (1997), and U.S. Pat. No. 5,739,386. Ortho-nitrobenzyl-based linkers, such as hydroxymethyl, hydroxyethyl, and Fmoc-aminoethyl carboxylic acid linkers, may also be obtained commercially. In some embodiments, ribonucleotides may be employed as cleavable nucleotides, wherein a cleavage step may be implemented using a ribonuclease, such as RNase H. In other embodiments, cleavage steps may be carried out by treatment with a nickase.

“Cluster” means an amplicon or clonal population of a single polynucleotide amplified by a surface amplification technique, such as bridge PCR. In some embodiments, the term “cluster” includes amplicons produced by rolling circle amplification.

“Hydrogel” means a gel comprising a crosslinked hydrophilic polymer network with the ability to absorb and retain large amounts of water (for example, 60 to 90 percent water, or 70 to 80 percent) without dissolution due to the establishment of physical or chemical bonds between the polymeric chains, which may be covalent, ionic or hydrogen bonds. Hydrogels exhibit high permeability to the oxygen and nutrients, making them attractive materials for cell encapsulation and culturing applications. Hydrogels may comprise natural or synthetic polymers and may be reversible (i.e. degradable or depolymerizable) or irreversible. Exemplary synthetic hydrogel polymers include polyethylene glycol (PEG), poly(2-hy droxyethyl methacrylate) and poly(vinyl alcohol). Exemplary natural hydrogel polymers include alginate, hyaluronic acid and collagen. The following reference describe hydrogels and their biomedical uses: Drury et al, Biomaterials, 24: 4337-4351 (2003); Garagorri et al, Acta Biomatter, 4(5): 1139-1147 (2008); Caliari et al, Nature Methods, 13(5): 405-414 (2016); Bowman et al, U.S. Pat. No. 9,631,092; Koh et al, Langmuir, 18(7): 2459-2462 (2002).

“On demand” means an operation may be directed to individual, discrete, selected locations (e.g. a spatial location of polymer precursor solution; or a selected polymer matrix chamber). Such selection may be based on manual observation of optical signals or data collected by a detector, or such selection may be based on a computer algorithm operating on optical signals or data collected by a detector. Manual observation of optical signals or data collected by a detector can include either real-time detection or detection at a time period prior to modulating a unit of energy to polymerize polymer precursors or degrading a chamber. For example, a subset of chambers (all formed with photo-degradable polymer matrix walls) may be pre-selected for releasing and removing their contents based on position information and the values of optical signals from an analytical assay carried out in the chambers. The pre-selected chambers may be photo-degraded by selectively projecting a light beam of appropriate wavelength characteristics (for example, with the spatial energy modulating element) to degrade the polymer matrix walls of the pre-selected chambers. In another example, a plurality of chambers may be observed in real-time (e.g. via fluorescent microscopy) for detection of an analyte of interest and one or more chambers of the plurality of chambers is selected, in real-time, upon detection of the analyte of interest, for degradation.

“Physical photomask” generally refers to a physical structure having a plurality of apertures or holes through which light may be projected. Physical photomasks can be used to create hydrogel matrices as described herein by causing the polymer precursor solution to polymerize and forming three-dimensional structures that correspond to the pattern on the photomask. A physical photomask can be patterned with a specific layout or geometric pattern. A physical photomask may be adhered to the upper surface of a flow cell.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, bridge PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” or “quantitative PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“tagman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference.

“Polymer matrix” generally refers to a phase material (e.g. continuous phase material) that comprises at least one polymer. In some embodiments, the polymer matrix refers to the at least one polymer as well as the interstitial space not occupied by the polymer. A polymer matrix may be composed of one or more types of polymers. A polymer matrix may include linear, branched, and crosslinked polymer units. A polymer matrix may also contain non-polymeric species intercalated within its interstitial spaces not occupied by polymer chains. The intercalated species may be solid, liquid, or gaseous species. For example, the term “polymer matrix” may encompass desiccated hydrogels, hydrated hydrogels, and hydrogels containing glass fibers. A polymer matrix may comprise a polymer precursor, which generally refers to one or more molecules that upon activation can trigger or initiate a polymeric reaction. A polymer precursor can be activated by electrochemical energy, photochemical energy, a photon, magnetic energy, or any other suitable energy. As used herein, the term “polymer precursor” includes monomers (that are polymerized to produce a polymer matrix) and crosslinking compounds, which may include photo-initiators, other compounds necessary or useful for generating polymer matrices, especially polymer matrices that are hydrogels.

“Polynucleotide” and “oligonucleotide” are used interchangeably and each means a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′ 3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

What is claimed is:

1. A method of making a spatially barcoded surface, the method comprising:
(a) disposing particles on a surface of a channel, wherein the surface is coated with primers for solid phase amplification, and wherein each particle comprises cleavably attached oligonucleotides comprising barcode sequences;
(b) synthesizing hydrogel chambers on the surface such that each hydrogel chamber encloses a particle; and
(c) cleaving the oligonucleotides from the particles so that the oligonucleotides are released into the chamber such that at least a portion of the oligonucleotides are captured by the primers, thereby obtaining captured oligonucleotides.

2. The method of claim 1, further comprising copying said captured oligonucleotides by extending said primers using said captured oligonucleotides as templates, thereby obtaining copies of said captured oligonucleotides.

3. The method of claim 2, further comprising amplifying said copies of said captured oligonucleotides to form clusters.

4. The method of claim 3, further comprising sequencing said barcode sequences of said copies of said captured oligonucleotides of said clusters.

5. The method of claim 4, wherein said copies of said captured oligonucleotides each comprise a sequencing primer binding site adjacent to said barcode sequence, and wherein said method further comprises determining said barcode sequence by a sequencing-by-synthesis method.

6. The method of claim 1, wherein said particles are disposed randomly on said surface.

7. The method of claim 1, wherein said particles are disposed in a regular pattern on said surface.

8. The method of claim 7, wherein said particles are disposed in a rectilinear pattern or a hexagonal pattern on said surface.

9. A method of making a spatially barcoded surface, the method comprising:
(a) providing a fluidic device comprising: (i) a channel comprising one or more polymer precursors and a surface comprising capture elements and a plurality of particles disposed on the surface, the particles having cleavably attached oligonucleotides comprising barcodes, (ii) a spatial energy modulation element in optical communication with the surface, and (iii) a detector in optical communication with the surface and in operable association with the spatial energy modulating element;
(b) using the detector, detecting each of the plurality of particles and identifying a position thereof on the surface;
(c) synthesizing one or more chambers in the channel, each chamber enclosing one or more of the plurality of particles, by projecting light into the channel with the spatial energy modulating element such that the projected light causes cross-linking of the one or more polymer precursors to form polymer matrix walls of the one or more chambers, wherein the polymer matrix walls are substantially impermeable to the oligonucleotides, and wherein positions of the synthesized chambers are determined by the positions of the particles enclosed thereby identified by the detector; and
(d) cleaving the oligonucleotides from the particles so that the oligonucleotides are released into the one or more chambers and are captured by the capture elements, thereby obtaining captured oligonucleotides.

10. The method of claim 9, further comprising copying said captured oligonucleotides by extending said capture elements using said captured oligonucleotides as templates, thereby obtaining copies of said captured oligonucleotides.

11. The method of claim 10, further comprising performing solid phase amplification of said copies of said captured oligonucleotides on said surface.

12. The method of claim 11, further comprising degrading said polymer walls of said one or more chambers.

13. A method of making a spatially barcoded surface, the method comprising one or more cycles of:
(a) disposing one or more particles on a surface of a channel, wherein the surface is coated with primers for solid phase amplification, and wherein each particle of the one or more particles comprises one or more cleavably attached oligonucleotides each comprising a barcode sequence;
(b) synthesizing hydrogel chambers on the surface such that each hydrogel chamber defines a sub-region of the surface and encloses a particle of the one or more particles;
(c) cleaving the one or more oligonucleotides from the one or more particles so that the one or more oligonucleotides are released into the hydrogel chambers such that at least a portion of the one or more oligonucleotides are captured by the primers and copied, thereby obtaining one or more copied oligonucleotides; and
(d) degrading the hydrogel chambers, thereby leaving the sub-regions each having attached thereto a copied oligonucleotide of the one or more copied oligonucleotides with a different barcode.

14. The method of claim 13, wherein said sub-regions do not overlap with one another.

15. The method of claim 14, further comprising amplifying said copied oligonucleotides, thereby obtaining amplified oligonucleotides.

16. The method of claim 15, wherein said barcode sequences are determined by sequencing said amplified oligonucleotides.

17. The method of claim 15, wherein a sum of areas of said sub-regions comprises at least 70 percent of an area of said surface.

18. The method of claim 17, wherein said sub-regions form a fractal barcode array.

18. The method of claim 13, wherein each of said sub-regions overlaps at least one other sub-region to form an overlap region.

19. The method of claim 18, wherein said surface in said overlap region comprises at least one oligonucleotide comprising two or more barcode sequences.

20. The method of claim 19, wherein relative positions of said sub-regions on said surface are determined by said oligonucleotides in said overlap regions which comprise two or more barcode sequences.
